(19) United States
(12) Patent Application Publication
(10) Pub. No.: US 2012/0323029 A1
(43) Pub. Date: Dec. 20, 2012

(54) Methods, Biological Oils, Biofuels, Units, and Organisms Related to Use in Compression Engines

(75) Inventors: Joseph W. Pfeifer, III, Westminster, MD (US); Paul W. Behrens, Ellicott City, MD (US); Michael A. Johnson, Laurel, MD (US); Jon M. Hansen, West Friendship, MD (US); Kirk E. Apt, Ellicott City, MD (US); William R. Barclay, Boulder, CO (US); David Christian Martin, Reisterstown, MD (US); Jacob Borden, Naperville, IL (US)

(73) Assignee: BP Biofuels IK Limited, Middlesex (GB)

(21) Appl. No.: 13/063,145

(22) PCT Filed: Mar. 8, 2011
(86) PCT No.: PCT/US11/27610

§ 371 Date: Aug. 4, 2011

Related U.S. Application Data
(60) Provisional application No. 61/313,055, filed on Mar. 11, 2010.

(51) Int. Cl. C12P 7/64; C12N 1/12; C07C 57/12; C12N 1/16

(52) U.S. Cl. 554/224; 435/255.1; 435/255.4; 435/257.3; 435/257.1; 435/255.3; 435/134

(57) ABSTRACT

Methods, biological oils, biofuels, units, and/or organisms directed to use in compression engines. A method of producing biological oils includes producing an organism and having the organism consume a feedstock. The organism includes a lipid containing fatty acids. The organism meets or exceeds at least two metrics. The metrics include: A) a cell density of at least about 115 grams per liter; B) a fatty acid content of at least about 49 percent on a dry mass basis; C) a fatty acid productivity of at least about 15 grams per liter per day; D) a fatty acid yield of at least about 0.175 grams of fatty acids produced per grams of the feedstock consumed; E) a 24 hour peak fatty acid productivity of at least about 30 grams per liter per day; F) an extraction efficiency on a percent of total fatty acid content basis of at least about 50 percent; and/or G) yield of fatty acids on oxygen of more than about 0.4 as grams of fatty acids produced per gram of oxygen consumed basis.

### CROSS-REFERENCE TO RELATED APPLICATIONS

This application claims the benefit of U.S. Provisional Patent Application No. 61/313,055, filed Mar. 11, 2010. The entire disclosure of U.S. Provisional Application No. 61/313,055 is hereby incorporated by reference into this specification in its entirety.

### NAMES OF THE PARTIES TO A JOINT RESEARCH AGREEMENT

For purposes of 35 U.S.C. §103(c)(2), a joint research agreement was executed between BP Biofuels UK Limited and Martek Biosciences Corporation on Dec. 18, 2008 in the field of biofuels. Also for the purposes of 35 U.S.C. §103(c)(2), a joint development agreement was executed between BP Biofuels UK Limited and Martek Biosciences Corporation on Aug. 7, 2009 in the field of biofuels.

### BACKGROUND

1. Technical Field

The invention is directed to methods, biological oils, biofuels, units, and/or organisms suitable for use in compression engines.

2. Discussion of Related Art

Issues of greenhouse gas levels and climate change have led to development of technologies seeking to utilize natural cycles between fixed carbon and liberated carbon dioxide. As these technologies advance, various techniques to convert feedstocks into biofuels have been developed. However, even with the above advances in technology, there remains a need and a desire to improve economic viability for conversion of renewable carbon sources to fuels.

### SUMMARY

The invention is directed to methods, biological oils, biofuels, units, and/or organisms suitable for use in compression engines. The methods of the invention provide an economic conversion of renewable carbon sources to fuels. Particularly, the invention includes the conversion of sugar containing carbon sources, such as crude extracts of sugar cane, to biodiesel through production of biological oil with an organism.

According to some embodiments, the invention is directed to a method of producing a biological oil containing fatty acids. The method includes producing and/or growing an organism where the organism includes fatty acids. Producing and/or growing can be accomplished simultaneously and/or sequentially. The organism meets or exceeds at least two metrics. The metrics include: A) a cell density of at least about 115 grams per liter; B) a fatty acid content of at least about 49 percent on a dry mass basis; C) a fatty acid productivity of at least about 15 grams per liter per day; D) a fatty acid yield of at least about 0.175 grams of fatty acids produced per grams of the feedstock consumed; E) a 24 hour peak fatty acid productivity of at least about 30 grams per liter per day, F) an extraction efficiency on a percent of total fatty acid content basis of at least about 50 percent; and/or G) a yield of fatty acids on oxygen expressed as grams of fatty acids produced per gram of oxygen consumed of at least about 0.4.

According to some embodiments, the invention is directed to a method of producing a biological oil containing fatty acids. The method includes producing and/or growing an organism where the organism includes fatty acids. Producing and/or growing can be accomplished simultaneously and/or sequentially. The organism meets or exceeds at least two metrics. The metrics include: a cell density of at least about 115 grams per liter; a fatty acid content of at least about 49 percent on a dry mass basis; a fatty acid productivity of at least about 15 grams per liter per day; a fatty acid yield of at least about 0.175 grams of fatty acids produced per grams of the feedstock consumed; a 24 hour peak fatty acid productivity of at least about 30 grams per liter per day; and/or a yield of fatty acids on oxygen expressed as grams of fatty acids produced per gram of oxygen consumed of at least about 0.4.

According to some embodiments, the organism meets or exceeds at least three of the metrics.

According to some embodiments, the organism meets or exceeds at least four of the metrics.

According to some embodiments, the organism meets or exceeds at least five of the metrics.

According to some embodiments, the organism consumes a feedstock, where the feedstock includes sucrose, glucose, fructose, xylose, glycerol, mannose, arabinose, lactose, galactose, maltose, or combinations thereof.

According to some embodiments, the feedstock includes a lignocellulosic derived material.

According to some embodiments, the organism includes organisms of a genus of Rhodosporidium, Pseudozyma, Tremella, Rhodotorula, Sporidiobolus, Sporobolomyces, Ustilago, Cryptococcus, Leucosporidium, Candida, or combinations thereof.

According to some embodiments, the organism includes organisms of a genus of Schizochytrium, Thraustochytrium, Ulkenia, Chlorella, Prototheca, or combinations thereof.

According to some embodiments, the organism includes Pseudozyma aphidis, Pseudozyma rugulosa, Pseudozyma sp., Rhodosporidium fluviale, Rhodosporidium paludigenum, Rhodotorula glutinis, Rhodotorula hordea, Sporobolomyces ruberrimus, Tremella sp., Ustilago sp., Rhodosporidium toruloides, Rhodotorula ingenosa, Sporidiobolus pararoseus, Leucosporidium scottii, Pseudozyma antarctica, Rhodosporidium sphaerocarpum, Rhodotorula muscorum, Cryptococcus laurentii, Candida tropicalis, Rhodosporidium diobovatum, Chlorella protothecoides, or combinations thereof.

According to some embodiments, the fatty acids include less than about 1 percent fatty acids with four or more double bonds on a mass basis.

According to some embodiments, the fatty acids include less than about 35 percent saturated fatty acids on a mass basis.

According to some embodiments, the fatty acids include a profile at least substantially similar to rapeseed.

According to some embodiments, the consuming and producing occur at a temperature of at least about 20 degrees Celsius.

According to some embodiments, the method further includes extracting the fatty acids from the organism, where the extracting has an efficiency of at least about 85 percent on a weight percent of total fatty acid content basis.

According to some embodiments, extracting includes solvent extraction with ethanol, hexane, an alcohol, or combinations thereof.

According to some embodiments, the fatty acid productivity meets or exceeds at least about 30 grams per liter per day and the fatty acid yield meets or exceeds at least about 0.175 grams of fatty acids produced per grams of feedstock consumed.

According to some embodiments, the 24 hour peak fatty acid productivity meets or exceeds at least about 50 grams per liter per day.

According to some embodiments, a 6 hour peak fatty acid productivity meets or exceeds at least about 70 grams per liter per day.

According to some embodiments, the consuming and producing occur under nitrogen limitation.

According to some embodiments, the feedstock includes at least one organic acid.

According to some embodiments, the consuming and producing occur at a pH of about 8 or below.

According to some embodiments, the method further includes converting the fatty acids to a biofuel by esterification, hydrogenation, or combinations thereof.

According to some embodiments, the invention is directed to a biological oil containing fatty acids made by any of the methods, units, and/or organisms disclosed within this specification.

According to some embodiments, the invention is directed to a biofuel made from the any of the biological oils disclosed within this specification.

According to some embodiments, the invention is directed to a biofuel suitable for use in a compression engine. The biofuel includes a fatty acid methyl ester profile having about 50 percent to about 70 percent oleic acid on a weight percent of total fatty acids basis, and/or about 15 percent to about 35 percent linolenic acid on weight percent of total fatty acids basis, where the biofuel is produced from a microorganism.

According to some embodiments, the fatty acid methyl ester profile derives from lipids produced by an organism from the kingdom stramenopile, the kingdom fungi, or combinations thereof.

According to some embodiments, the invention is directed to a unit for producing a biological oil. The unit includes a feedstock stream, a vessel connected to the feedstock stream, an organism disposed within the vessel, and a fatty acid containing stream connected to the vessel. The organism meets and/or exceeds at least two metrics. The metrics include: A) a cell density of at least about 115 grams per liter; B) a fatty acid content (intracellular and/or extracellular) of at least about 49 percent on a dry mass basis; C) a fatty acid productivity of at least about 15 grams per liter per day; D) a fatty acid yield of at least about 0.175 grams of fatty acids produced per grams of feedstock consumed; E) a 24 hour peak fatty acid productivity of at least about 30 grams per liter per day; F) an extraction efficiency on a percent of total fatty acid content basis of at least about 50 percent; and/or G) a yield of fatty acids on oxygen expressed as grams of fatty acids produced per gram of oxygen consumed of at least about 0.4.

According to some embodiments, the invention is directed to a unit for producing a biological oil. The unit includes a feedstock stream, a vessel connected to the feedstock stream, an organism disposed within the vessel, and a fatty acid containing stream connected to the vessel. The organism meets and/or exceeds at least two metrics. The metrics include: a cell density of at least about 115 grams per liter; a fatty acid content (intracellular and/or extracellular) of at least about 49 percent on a dry mass basis; a fatty acid productivity of at least about 15 grams per liter per day; a fatty acid yield of at least about 0.175 grams of fatty acids produced per grams of feedstock consumed; a 24 hour peak fatty acid productivity of at least about 30 grams per liter per day; and/or a yield of fatty acids on oxygen expressed as grams of fatty acids produced per gram of oxygen consumed of at least about 0.4.

According to some embodiments, the organism in the unit meets or exceeds at least three of the metrics.

According to some embodiments, the organism in the unit meets or exceeds at least four of the metrics.

According to some embodiments, the organism in the unit meets or exceeds at least five of the metrics.

According to some embodiments, the organism comprises an organism from kingdom stramenopile, kingdom fungi, or combinations thereof.

According to some embodiments, the vessel operates on a batch basis, a continuous basis, or combinations thereof.

According to some embodiments, the invention is directed to an isolated organism for producing a biological oil. The organism includes an overall index for fatty acid efficiency one (OILE1) of at least about 2.9. OILE1 has been defined as: OILE1=C*D*F, where C=a fatty acid productivity in grams per liter per day; D=a fatty acid yield in grams of fatty acid produced per grams of feedstock consumed; and F=an extraction efficiency on a percent of total fatty acid content basis.

According to some embodiments, the invention is directed to an isolated organism for producing a biological oil. The organism includes an overall index for fatty acid efficiency (OILE) of at least about 4,958. OILE has been defined as: OILE=A*B*C*D*E*F*G, where A=a cell density in grams per liter; B=a fatty acid content on a dry percent mass basis; C=a fatty acid productivity in grams per liter per day; D=a fatty acid yield in grams of fatty acids produced per grams of feedstock consumed; E=a 24 hour peak fatty acid productivity in grams per liter per day; F=an extraction efficiency on a percent of total fatty acid content basis; and G=a yield of fatty acids on oxygen expressed as grams of fatty acids produced per gram of oxygen consumed basis.

According to some embodiments, the invention is directed to an isolated organism for producing a biological oil. The organism includes an overall index for fatty acid efficiency two (OILE2) of at least about 235. OILE2 has been defined as: OILE2=A*B*C*D*F, where A=a cell density in grams per liter; B=a fatty acid content on a dry percent mass basis; C=a fatty acid productivity in grams per liter per day; D=a fatty acid yield in grams of fatty acids produced per grams of feedstock consumed; and F=an extraction efficiency on a percent of total fatty acid content basis.

According to some embodiments, the invention is directed to an isolated organism for producing a biological oil. The organism includes an overall index for fatty acid efficiency four (OILE4) of at least about 142. OILE4 has been defined as: OILE4=A*B*C*D*F*G, where A=a cell density in grams per liter; B=a fatty acid content on a dry percent mass basis; C=a fatty acid productivity in grams per liter per day; D=a fatty acid yield in grams of fatty acid produced per grams of feedstock consumed; F=an extraction efficiency on a percent of total fatty acid content basis; and G=a yield of fatty acid on oxygen expressed as grams of fatty acids produced per gram of oxygen consumed basis.

According to some embodiments, the isolated organism comprises naturally occurring organisms, genetically modified organisms, or combinations thereof.

According to some embodiments, the isolated organism consumes a carbon feedstock, where the carbon feedstock includes at least about 10 percent of total nitrogen consumed.

According to some embodiments, the isolated organism yields greater than about 50 percent more fatty acids when grown on sugar and glycerol as compared to primarily sugar alone.

According to some embodiments, the invention is directed to an isolated organism for producing a biological oil. The organism includes an overall index for fatty acid efficiency three (OILE3) of at least about 4.4. OILE3 has been defined as: OILE3=D*E*F, where D=a fatty acid yield in grams of fatty acids produced per grams of feedstock consumed; E=a 24 hour peak fatty acid productivity in grams per liter per day; and F=an extraction efficiency on a percent of total fatty acid content basis.

According to some embodiments, the invention is directed to a method of isolating an organism for producing a biological oil including identifying an organism with an overall index for fatty acid efficiency one (OILE1) of at least about 2.9 and isolating the organism. OILE1 has been defined as: OILE1=C*D*F, where C=a fatty acid productivity in grams per liter per day; D=a fatty acid yield in grams of fatty acid produced per grams of feedstock consumed; and F=an extraction efficiency on a percent of total fatty acid content basis.

According to some embodiments, the invention is directed to a method of isolating an organism for producing a biological oil including identifying an organism with an overall index for fatty acid efficiency (OILE) of at least about 4,958 and isolating the organism. OILE has been defined as: OILE=A*B*C*D*E*F*G, where A=a cell density in grams per liter; B=a fatty acid content on a dry percent mass basis; C=a fatty acid productivity in grams per liter per day; D=a fatty acid yield in grams of fatty acids produced per grams of feedstock consumed; E=a 24 hour peak fatty acid productivity in grams per liter per day; F=an extraction efficiency on a percent of total fatty acid content basis; and G=a yield of fatty acids on oxygen expressed as grams of fatty acids produced per gram of oxygen consumed basis.

According to some embodiments, the invention is directed to a method of isolating an organism for producing a biological oil including identifying an organism with an overall index for fatty acid efficiency two (OILE2) of at least about 235 and isolating the organism. OILE2 has been defined as: OILE2=A*B*C*D*F, where A=a cell density in grams per liter; B=a fatty acid content on a dry percent mass basis; C=a fatty acid productivity in grams per liter per day; D=a fatty acid yield in grams of fatty acids produced per grams of feedstock consumed; and F=an extraction efficiency on a percent of total fatty acid content basis.

According to some embodiments, the invention is directed to a method of isolating an organism for producing a biological oil including identifying an organism with an overall index for fatty acid efficiency four (OILE4) of at least about 142 and isolating the organism. OILE4 has been defined as: OILE4=A*B*C*D*F*G, where A=a cell density in grams per liter; B=a fatty acid content on a dry percent mass basis; C=a fatty acid productivity in grams per liter per day; D=a fatty acid yield in grams of fatty acid produced per grams of feedstock consumed; F=an extraction efficiency on a percent of total fatty acid content basis; and G=a yield of fatty acid on oxygen expressed as grams of fatty acids produced per gram of oxygen consumed basis.

According to some embodiments, the identified organism comprises naturally occurring organisms, genetically modified organisms, or combinations thereof.

According to some embodiments, the identified organism consumes a carbon feedstock, where the carbon feedstock includes at least about 10 percent of total nitrogen consumed.

According to some embodiments, the identified organism yields greater than about 50 percent more fatty acids when grown on sugar and glycerol as compared to primarily sugar alone.

According to some embodiments, the invention is directed to a method of isolating an organism for producing a biological oil including identifying an organism with an overall index for fatty acid efficiency three (OILE3) of at least about 4.4 and isolating the organism. OILE3 has been defined as: OILE3=D*E*F, where D=a fatty acid yield in grams of fatty acids produced per grams of feedstock consumed; E=a 24 hour peak fatty acid productivity in grams per liter per day; and F=an extraction efficiency on a percent of total fatty acid content basis.

According to some embodiments, the invention is directed to an isolated organism for producing a biological oil where the organism includes an overall index for fatty acid efficiency one (OILE1) of at least about 5.1. OILE1 has been defined as OILE1=C*D*F, where C=a fatty acid productivity in grams per liter per day, D=a fatty acid yield in grams of fatty acids produced per grams of feedstock consumed, and F=an extraction efficiency on a percent of total fatty acid content basis.

According to some embodiments, the isolated organism has a cell density of at least about 115 grams per liter.

According to some embodiments, the isolated organism has a fatty acid content of at least about 49 percent on a dry mass basis.

According to some embodiments, the isolated organism has a 24 hour peak fatty acid productivity of at least about 30 grams per liter per day.

According to some embodiments, the isolated organism has a yield of fatty acid on oxygen of more than about 0.4 grams of fatty acids produced per gram of oxygen consumed.

According to some embodiments, the isolated organism is a fungus, an algae, and/or combinations thereof.

According to some embodiments, the isolated organism comprises organisms of a genus of Rhodosporidium, Pseudozyma, Tremella, Rhodotorula, Sporidiobolus, Sporobolomyces, Ustilago, Cryptococcus, Leucosporidium, Candida, or combinations thereof.

According to some embodiments, the isolated organism comprises organisms of a genus of Schizochytrium, Thraustochytrium, Ulkenia, Chlorella, Prototheca, or combinations thereof.

According to some embodiments, the isolated organism comprises Pseudozyma aphidis, Pseudozyma rugulosa, Pseudozyma sp., Rhodosporidium fluviale, Rhodosporidium paludigenum, Rhodotorula glutinis, Rhodotorula hordea, Rhodotorula ingenosa, Sporobolomyces ruberrimus, Tremella sp., Ustilago sp., Rhodosporidium toruloides, Sporidiobolus pararoseus, Leucosporidium scottii, Pseudozyma antarctica, Rhodosporidium sphaerocarpum, Rhodotorula muscorum, Cryptococcus laurentii, Candida tropicalis, Rhodosporidium diobovatum, Chlorella protothecoides, or combinations thereof.

According to some embodiments, the isolated organism comprises Pseudozyma aphidis, Pseudozyma rugulosa, Rhodotorula ingenosa, Sporidiobolus pararoseus, or combinations thereof.

According to some embodiments, the isolated organism comprises Pseudozyma aphidis.

According to some embodiments, the isolated organism comprises Pseudozyma rugulosa.

According to some embodiments, the isolated organism comprises Rhodotorula ingenosa.

According to some embodiments, the isolated organism comprises Sporidiobolus pararoseus.

According to some embodiments, isolated organism has the identifying characteristics of ATCC Patent Deposit Designation of PTA-11615 and mutant strains derived therefrom.

According to some embodiments, isolated organism has the identifying characteristics of ATCC Patent Deposit Designation of PTA-11615.

According to some embodiments, isolated organism has the identifying characteristics of ATCC Patent Deposit Designation of PTA-11615, and derivatives, variants, and/or mutant strains derived therefrom.

According to some embodiments, the isolated organism has the identifying characteristics of ATCC Patent Deposit Designation of PTA-11616 and mutant strains derived therefrom.

According to some embodiments, the isolated organism has the identifying characteristics of ATCC Patent Deposit Designation of PTA-11616.

According to some embodiments, the isolated organism has the identifying characteristics of ATCC Patent Deposit Designation of PTA-11616, and derivatives, variants, and/or mutant strains derived therefrom.

According to some embodiments, the isolated organism has the identifying characteristics of ATCC Patent Deposit Designation of PTA-11617 and mutant strains derived therefrom.

According to some embodiments, the isolated organism has the identifying characteristics of ATCC Patent Deposit Designation of PTA-11617.

According to some embodiments, the isolated organism has the identifying characteristics of ATCC Patent Deposit Designation of PTA-11617, and derivatives, variants, and/or mutant strains derived therefrom.

According to some embodiments, the isolated organism has an overall index for fatty acid efficiency (OILE) of at least about 4,958. Where OILE has been defined as OILE=A*B*C*D*E*F*G, A=a cell density in grams per liter, B=a fatty acid content on a dry percent mass basis, C=a fatty acid productivity in grams per liter per day, D=a fatty acid yield in grams of fatty acids produced per grams of feedstock consumed, E=a 24 hour peak fatty acid productivity in grams per liter per day, F=an extraction efficiency on a percent of total fatty acid content basis, and G=a yield of fatty acid on oxygen on a grams of total fatty acids per gram of oxygen consumed basis.

According to some embodiments, the invention includes a biological oil comprising fatty acids made from an isolated organism disclosed herein.

According to some embodiments, the invention includes biofuel made from a biological oil disclosed herein.

According to some embodiments, the invention includes method of producing a biological oil by producing an organism comprising fatty acids, and removing the fatty acids from the organism to form the biological oil. Where the organism meets or exceeds at least two metrics. The metrics include a cell density of at least about 115 grams per liter, a fatty acid content of at least about 49 percent on a dry mass basis, a fatty acid productivity of at least about 15 grams per liter per day, a fatty acid yield of at least about 0.175 grams of fatty acids produced per grams of feedstock consumed, a 24 hour peak fatty acid productivity of at least about 30 grams per liter per day, and/or a yield of fatty acid on oxygen of more than about 0.4 grams of fatty acids produced per gram of oxygen consumed.

According to some embodiments, the organism of the method meets or exceeds at least one more of the metrics.

According to some embodiments, the organism of the method meets or exceeds at least two more of the metrics.

According to some embodiments, the organism of the method meets or exceeds at least three more of the metrics.

According to some embodiments, the organism of the method comprises organisms of a genus of Rhodosporidium, Pseudozyma, Tremella, Rhodotorula, Sporidiobolus, Sporobolomyces, Ustilago, Cryptococcus, Leucosporidium, Candida, or combinations thereof.

According to some embodiments, the organism of the method comprises organisms of a genus of Schizochytrium, Thraustochytrium, Ulkenia, Chlorella, Prototheca, or combinations thereof.

According to some embodiments, the organism comprises Pseudozyma aphidis, Pseudozyma rugulosa, Pseudozyma sp., Rhodosporidium fluviale, Rhodosporidium paludigenum, Rhodotorula glutinis, Rhodotorula hordea, Rhodotorula ingenosa, Sporobolomyces ruberrimus, Tremella sp., Ustilago sp., Rhodosporidium toruloides, Sporidiobolus pararoseus, Leucosporidium scottii, Pseudozyma antarctica, Rhodosporidium sphaerocarpum, Rhodotorula muscorum, Cryptococcus laurentii, Candida tropicalis, Rhodosporidium diobovatum, Chlorella protothecoides, or combinations thereof.

According to some embodiments, the fatty acids of the method comprise less than about 1 percent fatty acids with four or more double bonds on a mass basis.

According to some embodiments, the fatty acids of the method comprise less than about 35 percent saturated fatty acids on a mass basis.

According to some embodiments, a 6 hour peak fatty acid productivity meets or exceeds at least about 70 grams per liter per day.

According to some embodiments, the method includes consuming a feedstock, where the feedstock comprises at least one organic acid.

According to one embodiment, the biological oil comprising fatty acids made by any of the methods disclosed herein.

According to one embodiment, the invention includes a biofuel made from any of the biological oils disclosed herein.

According to one embodiment, the invention includes a biofuel suitable for use in a compression engine. The biofuel comprising a fatty acid methyl ester profile of about 50 percent to about 70 percent oleic acid on a weight percent of total fatty acids basis, about 15 percent to about 35 percent linoleic acid on a weight percent of total fatty acids basis, and about less than about 10 percent palmitic acid on a weight percent of total fatty acid basis. Where the biofuel is produced from an oleaginous microorganism.

According to one embodiment, the fatty acid methyl ester profile derives from lipids produced by an organism from the kingdom stramenopile, the kingdom fungi, or combinations thereof.

According to some embodiments, the invention is directed to an engine operating on a biofuel made from the any of the biological oils disclosed within this specification.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 schematically shows a unit, according to some embodiments;

FIG. 2 schematically shows a two stage unit, according to some embodiments;

FIG. 3 schematically shows a unit with extraction, according to some embodiments;

FIG. 4 schematically shows a fermentor, according to some embodiments;

FIG. 5 shows a graph of time versus biomass, according to some embodiments; and

FIG. 6 shows a graph of time versus fat, according to some embodiments.

### DETAILED DESCRIPTION

The invention is directed to methods, biological oils, biofuels, units, and/or isolated organisms suitable for use in compression engines. According to some embodiments, the invention can include an improved production of biological oil and/or oils. The invention can also include production of microbial lipids and production of biodiesel using the microbial fatty acids contained in those lipids.

According to some embodiments, the invention includes developing technology for the very low cost production of microbial oils that are suitable for biodiesel production and/or nutritional applications. Lower production costs for microbial oils can be made using a holistic approach to the problem of producing microbial oils. Efforts primarily focused on one or two aspects of the problem alone may not be able to see key hurdles to be addressed very early in the technology development of microbial oils.

The invention can include a targeted production process and/or model with an expertise in microbial oils, an understanding in fuels, and/or the like. The targeted production process can identify the key hurdles to overcome along the way to producing very low cost microbial oils. Information can be used in a multivariate approach and/or analysis to guide development of key components of technology including strain isolation, strain development, fermentation equipment design, fermentation operating modes, fermentation strategies, and/or the like.

According to some embodiments, an integrated approach results in methods and/or processes utilizing complex low cost carbon substrates to produce microbial oils. Strains of organisms were isolated and/or developed to produce large amounts of oil with a desirable fatty acid profile while growing on the targeted substrates under targeted production conditions. The targeted production conditions included high temperatures, low chloride levels, and/or the like. The strains of the organisms were able to grow using targeted low cost fermentation equipment and utilize low cost extraction technologies to remove the oil from the strain of the organism.

The integrated approach enables simultaneous achievement of multiple parameters: 1) higher levels of cell concentration, 2) higher fatty acid content, 3) better fatty acid yield per unit sugar consumed, 4) higher fatty acid productivity and/or 5) lower oxygen requirement per quantity of fatty acids produced than previously achieved. The five key parameters and/or variables can produce very low cost microbial oils, such as microbial oils suitable for use in production of biodiesel. Economically viable biodiesel can compete with petroleum based diesel on costs, desirably, with and/or without government mandates and/or subsidies. Biodiesel also can compete with and/or exceed petroleum based diesel on performance criteria, such as cetane, cold flow properties, cloud point, soot formation, particulates, sulfur content, nitrogen oxide generation, and/or the like.

The integrated approach can produce microbial lipids with a unique fatty acid profile, such as a rapeseed-like profile. Meeting the five parameters can be done by utilizing novel strains isolated with targeted attributes, identifying key fermentation parameters, determining conditions or processes that improve fatty acid production, efficiently utilizing carbon and other nutrients from low cost substrates or sources, and/or the like. The integrated approach addresses key problems not previously remedied for very low cost production of microbial oils, such as involved in economic production of biodiesel.

Examples of the integrated approach can include, but are not limited to, the following items: (1) identification of genera and strains of yeast that were not known previously to be good oil producing types of microorganisms and that the genera and strains could produce oil on the targeted substrates, such as sucrose and xylose; (2) identification and solution of problems with oil producing yeast and utilization of sucrose or xylose as a carbon source; (3) an ability to significantly improve fatty acid yield on sucrose by increasing fermentation temperature, decreasing pH, and decreasing nitrogen levels.

For example, under nitrogen limiting conditions (favoring oil production) yeast can reduce and/or stop utilizing a fructose portion of sucrose (glucose+fructose). Surprisingly and unexpectedly, glycerol addition to a fermentation medium allows the yeast to start utilizing fructose from the carbon source, such as sucrose. Similarly, increasing fermentation temperature, decreasing pH, and decreasing nitrogen levels can favorably lower production cost while increasing oil production. In other embodiments, at least 10 percent of total nitrogen, at least about 20 percent total nitrogen, at least about 40 percent of total nitrogen, and/or at least about 50 percent of total nitrogen can be supplied to the fermentation as part of the carbon source feedstock during the production of an oil.

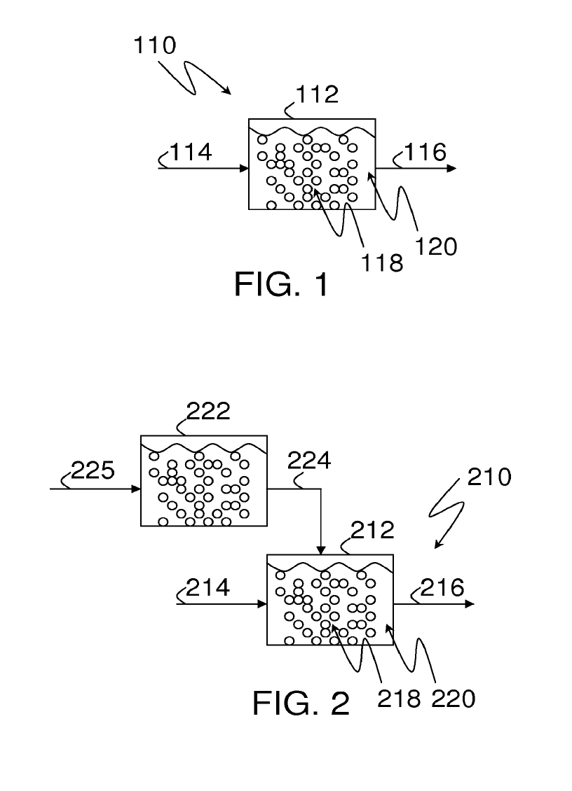

FIG. 1 schematically shows a unit 110, according to some embodiments. In the Figures, like reference numerals are used to indicate identical or functionally similar elements. The left most digit of each reference numeral corresponds to the Figure in which the reference numeral first appears. The unit 110 includes a vessel 112 with a feedstock stream 114 connected to the vessel 112 and a lipid (fatty acids) stream 116 connected to the vessel 112. The feedstock stream 114 provides feedstock to the vessel 112, wherein the feedstock can be any of the materials and/or substances included in the definition of feedstock below. Lipids present in the vessel 112 exit the vessel 112 through the lipid stream 116, wherein the lipids can be any of the substances included in the definition of lipids below. The vessel 112 includes or contains an organism 18 disposed within the vessel 112, wherein the organism 118 can be any of the substances included in the definition of organism below. The vessel 112 includes or contains a medium 120, such as a fermentation broth. The organism 118 can be in the medium 120.

FIG. 2 schematically shows a two stage unit 210, according to some embodiments. The two stage unit 210 includes a vessel 212 with a feedstock stream 214 connected to the vessel 212 and a lipid (fatty acids) stream 216 connected to the vessel 212. The feedstock stream 214 provides feedstock to the vessel 212, wherein the feedstock can be any of the materials and/or substances included in the definition of feedstock below. Lipids present in the vessel 212 exit the vessel 212 through the lipid stream 216, wherein the lipids can be any of the substances included in the definition of lipids below. The vessel 212 includes or contains an organism 218 disposed within the vessel 212, wherein the organism 218 can be any of the substances included in the definition of organism below. The vessel 212 includes or contains a medium 220, such as a fermentation broth. The organism 218 can be in the medium 220. The two stage unit 210 includes a growth vessel 222 with a growth feedstock stream 225 and an organism stream 224 that connects the growth vessel 222 to the vessel 212. The growth feedstock stream 225 provides growth feedstock to the growth vessel 222, wherein the growth feedstock can be the same feedstock present in the feedstock stream 214. The organism stream 224 provides the organism 218 from the growth vessel 222 to the vessel 212.

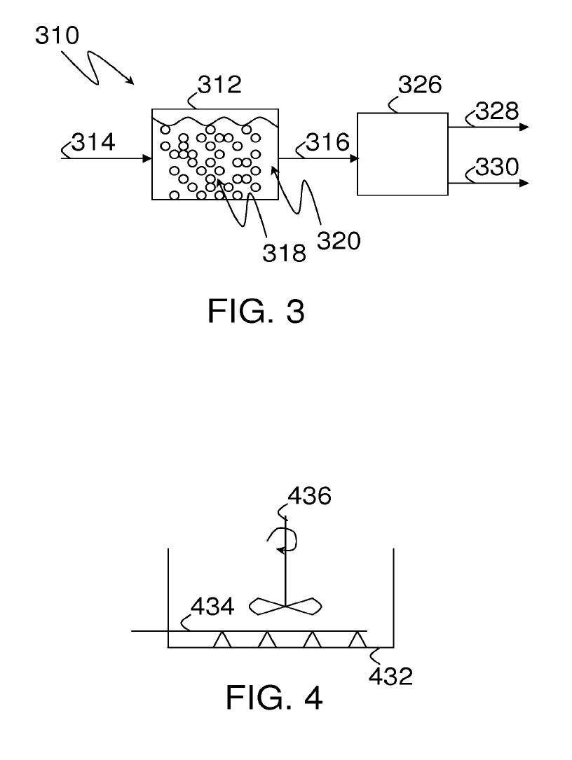

FIG. 3 schematically shows a unit 310 with extraction, according to some embodiments. The unit 310 includes a vessel 312 with a feedstock stream 314 connected to the vessel 312 and a lipid (fatty acids) stream 316 connected to the vessel 312. The feedstock stream 314 provides feedstock to the vessel 312, wherein the feedstock can be any of the materials and/or substances included in the definition of feedstock below. Lipids present in the vessel 312 exit the vessel 312 through the lipid stream 316, wherein the lipids can be any of the substances included in the definition of lipids below. The vessel 312 includes or contains an organism 318 disposed within the vessel 312, wherein the organism 218 can be any of the substances included in the definition of organism below. The vessel 312 includes or contains a medium 320, such as a fermentation broth. The organism 318 can be in the medium 320. The unit 310 includes an extraction apparatus 326. The lipid stream 316 is fed to the extraction apparatus 326 from the vessel 312. The extraction apparatus 326 removes the lipids present in lipid stream 316 from the remainder of the contents of the lipid stream 316 so that a lipid product stream 328 exits the extraction apparatus 326. A delipidated biomass stream 330 also exits the extraction apparatus 326.

FIG. 4 schematically shows a fermentor 432, according to some embodiments. The fermentor 432 includes a sparger 434, such as for introduction of air and/or other gases into a process. The fermentor 432 includes an agitator 436, such as for stirring contents of the fermentor 432. In some embodiments, the vessel 112, the vessel 212, or the vessel 312 can be a fermentor similar to the fermentor 432.

According to some embodiments, the invention can include a method of producing biological oils. The method can include producing or growing an organism. The method can include meeting or exceeding at least two metrics. The organism can include and/or have within a lipid containing fatty acids and/or a quantity of lipids containing fatty acids. In the alternative, the organism can excrete and/or discharge the biological oil. The metrics can include:
A) a cell density of at least about 115 grams per liter;
B) a fatty acid content of at least about 49 percent on a dry mass basis;
C) a fatty acid productivity of at least about 15 grams per liter per day;
D) a fatty acid yield of at least about 0.175 grams of fatty acid produced per grams of feedstock consumed;
E) a 24 hour peak fatty acid productivity of at least about 30 grams per liter per day;
F) an extraction efficiency on a percent fatty acid content basis of at least about 50 percent; and/or
G) a yield of fatty acid on oxygen expressed as grams of fatty acids produced per gram of oxygen consumed of at least about 0.4.
   Any suitable combination of metrics can satisfy a desired criteria, such as: A and B; A and C; A and D; A and E; A and F; A and G; B and C; B and D; B and E; B and F; B and G; C and D; C and E; C and F; C and G; D and E; D and F; D and G; E and F; E and G; F and G; A, B, and C; A, B, and D; A, B, and E; A, B, and F; A, B, and G; B, C, and D; B, C, and E; B, C, and F; B, C, and G; C, D, and E; C, D, and F; C, D, and G; D, E, and F; D, E, and G; E, F, and G; A, B, C, and D; A, B, C, and E; A, B, C, and F; A, B, C, and G; B, C, D, and E; B, C, D, and F; B, C, D, and G; C, D, E, and F; C, D, E, and G; D, E, F, and G; A, B, C, D, and E; A, B, C, D, and F; A, B, C, D, and G; B, C, D, E, and F; B, C, D, E, and G; C, D, E, F, and G; A, B, C, D, E, and F; A, B, C, D, E, and G; B, C, D, E, F, and G; A, B, C, D, E, F, and G; and/or the like.

In the alternative, the method can include consuming a feedstock to produce an organism.

Producing and production refers to making, forming, creating, shaping, bringing about, bringing into existence, manufacturing, growing, synthesizing, and/or the like. According to some embodiments, producing includes fermentation, cell culturing, and/or the like. Producing can include new or additional organisms as well as maturation of existing organisms.

Growing refers to increasing in size, such as by assimilation of material into the living organism and/or the like.

Biological refers to life systems, living processes, alive organisms, and/or the like. Biological can refer to organisms from archaea, bacteria, and/or eukarya. Biological can also refer to derived and/or modified compounds and/or materials from biological organisms. According to some embodiments, biological excludes fossilized and/or ancient materials, such as those whose life ended at least about 1,000 years ago.

Oil refers to hydrocarbon substances and/or materials that are at least somewhat hydrophobic and/or water repelling. Oil can include mineral oil, organic oil, synthetic oil, essential oil, and/or the like. Mineral oil refers to petroleum and/or related substances derived at least in part from the Earth and/or underground, such as fossil fuels. Organic oil refers to materials and/or substances derived at least in part from plants, animals, other organisms, and/or the like. Synthetic oil refers to materials and/or substances derived at least in part from chemical reactions and/or processes, such as can be used in lubricating oil. Oil can be at least generally soluble in nonpolar solvents and other hydrocarbons, but at least generally insoluble in water and/or aqueous solutions. Oil can be at least about 50 percent soluble in nonpolar solvents, at least about 75 percent soluble in nonpolar solvents, at least about 90 percent soluble in nonpolar solvents, completely soluble in nonpolar solvents, about 50 percent soluble in nonpolar solvents to about 100 percent soluble in nonpolar solvents and/or the like, all on a mass basis.

Lipid refers to oils, fats, waxes, greases, cholesterol, glycerides, steroids, phosphatides, cerebrosides, fatty acids, fatty acid related compounds, derived compounds, other oily substances, and/or the like. Lipids can be made in living cells and can have a relatively high carbon content and a relatively high hydrogen content with a relatively lower oxygen content. Lipids typically include a relatively high energy content, such as on a mass basis.

Biological oils refer to hydrocarbon materials and/or substances derived at least in part from living organisms, such as animals, plants, fungi, yeasts, algae, microalgae, bacteria, and/or the like. According to some embodiments, biological oils can be suitable for use as and/or conversion into renewable materials and/or biofuels.

Renewable materials refer to substances and/or items that have been at least partially derived from a source and/or process capable of being replaced by natural ecological cycles and/or resources. Renewable materials can include chemicals, chemical intermediates, solvents, monomers, oligomers, polymers, biofuels, biofuel intermediates, biogasoline, biogasoline blendstocks, biodiesel, green diesel, renewable diesel, biodiesel blend stocks, biodistillates, and/or the like. In some embodiments, the renewable material can be derived from a living organism, such as plants, algae, bacteria, fungi, and/or the like.

Biofuel refers to components and/or streams suitable for use as a fuel and/or a combustion source derived at least in part from renewable sources. The biofuel can be sustainably produced and/or have reduced and/or no net carbon emissions to the atmosphere, such as when compared to fossil fuels. According to some embodiments, renewable sources can exclude materials mined or drilled, such as from the underground. In some embodiments, renewable resources can include single cell organisms, multicell organisms, plants, fungi, bacteria, algae, cultivated crops, noncultivated crops, timber, and/or the like. Biofuels can be suitable for use as transportation fuels, such as for use in land vehicles, marine vehicles, aviation vehicles, and/or the like. Biofuels can be suitable for use in power generation, such as raising steam, exchanging energy with a suitable heat transfer media, generating syngas, generating hydrogen, making electricity, and or the like.

Biodiesel refers to components or streams suitable for direct use and/or blending into a diesel pool and/or a cetane supply derived from renewable sources. Suitable biodiesel molecules can include fatty acid esters, monoglycerides, diglycerides, triglycerides, lipids, fatty alcohols, alkanes, naphthas, distillate range materials, paraffinic materials, aromatic materials, aliphatic compounds (straight, branched, and/or cyclic), and/or the like. Biodiesel can be used in compression ignition engines, such as automotive diesel internal combustion engines, truck heavy duty diesel engines, and/or the like. In the alternative, the biodiesel can also be used in gas turbines, heaters, boilers, and/or the like. According to some embodiments, the biodiesel and/or biodiesel blends meet or comply with industrially accepted fuel standards, such as B20, B40, B60, B80, 699.9, B100, and/or the like.

Biodistillate refers to components or streams suitable for direct use and/or blending into aviation fuels (jet), lubricant base stocks, kerosene fuels, fuel oils, and/or the like. Biodistillate can be derived from renewable sources, and have any suitable boiling point range, such as a boiling point range of about 100 degrees Celsius to about 700 degrees Celsius, about 150 degrees Celsius to about 350 degrees Celsius, and/or the like.

Consuming refers to using up, utilizing, eating, devouring, transforming, and/or the like. According to some embodiments, consuming can include processes during and/or a part of cellular metabolism (catabolism and/or anabolism), cellular respiration (aerobic and/or anaerobic), cellular reproduction, cellular growth, fermentation, cell culturing, and/or the like.

Feedstock refers to materials and/or substances used to supply, feed, provide for, and/or the like, such as to an organism, a machine, a process, a production plant, and/or the like. Feedstocks can include raw materials used for conversion, synthesis, and/or the like. According to some embodiments, the feedstock can include any material, compound, substance, and/or the like suitable for consumption by an organism, such as sugars, hexoses, pentoses, monosaccharides, disaccharides, trisaccharides, polyols (sugar alcohols), organic acids, starches, carbohydrates, and/or the like. According to some embodiments, the feedstock can include sucrose, glucose, fructose, xylose, glycerol, mannose, arabinose, lactose, galactose, maltose, other five carbon sugars, other six carbon sugars, other twelve carbon sugars, plant extracts containing sugars, other crude sugars, and/or the like. Feedstock can refer to one or more of the organic compounds listed above when present in the feedstock.

According to some embodiments, the method and/or process can include addition of other materials and/or substances to aid and/or assist the organism, such as nutrients, vitamins, minerals, metals, water, and/or the like. The use of other additives are also within the scope of this invention, such as antifoam, flocculants, emulsifiers, demulsifiers, viscosity increases, viscosity decreasers, surfactants, salts, other fluid modifying materials, and/or the like.

Organic refers to carbon containing compounds, such as carbohydrates, sugars, ketones, aldehydes, alcohols, lignin, cellulose, hemicellulose, pectin, other carbon containing substances, and/or the like.

According to some embodiments, the feedstock can be fed into the fermentation using one or more feeds. In some embodiments, feedstock can be present in media charged to a vessel prior to inoculation. In some embodiments, feedstock can be added through one or more feed streams in addition to the media charged to the vessel.

According to some embodiments, the feedstock can include a lignocellulosic derived material, such as material derived at least in part from biomass and/or lignocellulosic sources. Biomass refers to plant and/or animal materials and/or substances derived at least in part from living organisms and/or recently living organisms, such as plants and/or lignocellulosic sources. Biomass can include other materials and/or substances to aid and/or assist the organism, such as nutrients, vitamins, minerals, metals, water, and/or the like.

Lignocellulosic refers to containing at least some cellulose, hemicellulose, lignin, and/or the like. Lignocellulosic can refer to plant and/or plant derived material. Lignocellulosic material can include any suitable material, such as sugar cane, sugar cane bagasse, energy cane, energy cane bagasse, rice, rice straw, corn, corn stover, wheat, wheat straw, maize, maize stover, sorghum, sorghum stover, sweet sorghum, sweet sorghum stover, cotton, cotton remnant, cassava, sugar beet, sugar beet pulp, soybean, rapeseed, jatropha, switchgrass, miscanthus, other grasses, timber, softwood, hardwood, wood bark, wood waste, sawdust, paper, paper waste, agricultural waste, manure, dung, sewage, municipal solid waste, any other suitable biomass material, and/or the like. Lignocellulosic material can be pretreated and/or treated by any suitable process and/or method, such as acid hydrolysis, neutral hydrolysis, basic hydrolysis, thermal hydrolysis, catalytic hydrolysis, enzymatic hydrolysis, ammonia fiber expansion, steam explosion, and/or the like.

Cell culturing refers to metabolism of carbohydrates whereby a final electron donor is oxygen, such as aerobically. Cell culturing processes can use any suitable organisms, such as bacteria, fungi (including yeast), algae, and/or the like. Suitable cell culturing processes can include naturally occurring organisms and/or genetically modified organisms.

Fermentation refers both to cell culturing and to metabolism of carbohydrates where a final electron donor is not oxygen, such as anaerobically. Fermentation can include an enzyme controlled anaerobic breakdown of an energy rich compound, such as a carbohydrate to carbon dioxide and an alcohol, an organic acid, a lipid, and/or the like. In the alternative, fermentation refers to biologically controlled transformation of an inorganic or organic compound. Fermentation processes can use any suitable organisms, such as bacteria, fungi (including yeast), algae, and/or the like. Suitable fermentation processes can include naturally occurring organisms and/or genetically modified organisms.

Biological processes can include any suitable living system and/or item derived from a living system and/or a process. Biological processes can include fermentation, cell culturing, aerobic respiration, anaerobic respiration, catabolic reactions, anabolic reactions, biotransformation, saccharification, liquefaction, hydrolysis, depolymerization, polymerization, and/or the like.

Organism refers to an at least relatively complex structure of interdependent and subordinate elements whose relations and/or properties can be largely determined by their function in the whole. The organism can include an individual designed to carry on the activities of life with organs separate in function but mutually dependent. Organisms can include a living being, such as capable of growth, reproduction, and/or the like.

The organism can include any suitable simple (mono) cell being, complex (multi) cell being, and/or the like. Organisms can include algae, fungi (including yeast), bacteria, and/or the like. The organism can include microorganisms, such as bacteria or protozoa. The organism can include one or more naturally occurring organisms, one or more genetically modified organisms, combinations of naturally occurring organisms and generically modified organisms, and/or the like. Embodiments with combinations of multiple different organisms are within the scope of the invention. Any suitable combination or organism can be used, such as one or more organisms, at least about two organisms, at least about five organisms, about two organisms to about twenty organisms, and/or the like.

Oleaginous refers to oil bearing, oil containing and/or producing oils, lipids, fats, and/or other oil-like substances. Oleaginous may include organisms that produce at least about 20 percent by weight of oils, at least about 30 percent by weight of oils, at least about 40 percent by weight oils, at least about 50 percent by weight oils, at least about 60 percent by weight oils, at least about 70 percent by weight oils, at least about 80 percent by weight oils, and/or the like.

According to some embodiments, the organism can include a member of the kingdom stramenopile, such as a thraustochytrid and/or golden algae, for example. The organism can be of the genus Schizochytrium, Thraustochytrium, Ulkenia, and/or the like.

According to some embodiments, the organism can include a member of the kingdom planta. The organism can be of the genus Chlorella, Prototheca, and/or the like.

In the alternative, the organism can be a single cell member of the fungal kingdom, such as a yeast, for example. The organism can be of the genus Rhodosporidium, Leucosporidium, Pseudozyma, Tremella, Rhodotorula, Sporidiobolus, Sporobolomyces, Ustilago, Cryptococcus, Leucosporidium, Candida, and/or the like.

According to some embodiments, the organism can include Pseudozyma aphidis, Pseudozyma rugulosa, Pseudozyma sp., Rhodosporidium fluviale, Rhodosporidium paludigenum, Rhodotorula glutinis, Rhodotorula hordea, Rhodotorula ingenosa, Sporobolomyces ruberrimus, Tremella sp., Ustilago sp., Rhodosporidium toruloides including CBS 6016, Rhodosporidium toruloides including CBS 8587, Sporidiobolus pararoseus, Leucosporidium scottii, Pseudozyma antarctica, Rhodosporidium sphaerocarpum, Rhodotorula muscorum, Cryptococcus laurentii, Candida tropicalis, Rhodosporidium diobovatum, Chlorella protothecoides including UTEX 250, and/or the like.

According to some embodiments utilizing sucrose, the organism can include Leucosporidium scottii, Pseudozyma Antarctica, Rhodosporidium sphaerocarpum, Rhodotorula muscorum, and/or the like. According to some embodiments utilizing xylose, the organism can include Leucosporidium scottii, Cryptococcus laurentii, Candida tropicalis, Rhodosporidium diobovatum, Rhodosporidium toruloides, Pseudozyma Antarctica, Sporidiobolus pararoseus, Rhodotorula muscorum, and/or the like.

The organism can operate, function, and/or live under any suitable conditions, such as anaerobically, aerobically, photosynthetically heterotrophically, and/or the like.

Genetic engineering refers to intentional manipulation and/or modification of at least a portion of a genetic code and/or expression of a genetic code of an organism.

Genetically modified refers to organisms, cultures, single cells, biota, and/or the like that have been genetically engineered. Genetically modified organisms can include those manipulated by genomic mutagenesis, addition and/or removal of one or more genes, portions of proteins, promoter regions, noncoding regions, chromosomes, and/or the like.

Naturally occurring refers to organisms, cultures, single cells, biota, and/or the like at least generally without intervening actions by exterior forces, such as humankind, machine, and/or the like. Naturally occurring organisms can include those found in local environments (flora and/or fauna) and/or the like. Naturally occurring organisms can be collected, isolated, cultured, purified, and/or the like.

According to some embodiments, the organism can include an ability to yield greater than about 25 percent, greater than about 50 percent, greater than about 75 percent, about 100 percent, about 45 percent to about 90 percent, and/or the like of dry weight of the organism as fatty acids and yield an equivalent and/or a better amount of biomass when grown on xylose, sucrose, and/or glycerol as compared to a yield on primarily glucose alone.

According to some embodiments, the organism can yield greater than about 25 percent, greater than about 50 percent, greater than about 75 percent, about 100 percent, about 25 percent to about 100 percent, and/or the like more fatty acids when grown on a combination of primarily sugar (e.g., sucrose, glucose, fructose, xylose, and/or the like) and glycerol as compared to primarily the sugar alone. The ratio of sugar to glycerol can be any suitable amount, such as about 100:1, about 50:1, about 10:1, about 1:1, about 1:10, about 1:50, about 1:100, about 1-20:50-100, and/or the like on, a mass basis, a mole basis, a volume basis, and/or the like.

In some embodiments, producing an organism includes where the organism includes fatty acids and/or results in an organism containing fatty acids, such as within or on one or more vesicles and/or pockets. In the alternative, the fatty acid can be relatively uncontained within the cell and/or outside the cell, such as relatively free from constraining membranes. Producing the organism can include cellular reproduction (more cells) as well as cell growth (increasing a size and/or contents of the cell, such as by increasing a fatty acid content). Reproduction and growth can occur at least substantially simultaneously with each other, at least substantially exclusively of each other, at least partially simultaneously and at least partially exclusively, and/or the like.

According to some embodiments, the method can include at least substantially simultaneously meeting or exceeding at least two or more of the metrics (A-G) listed above. An economically viable biological oil method for biofuels can compete with other energy sources, such as crude oil. Accordingly, several factors can be used to deliver an economic process. Surprisingly and unexpectedly, a method of producing biological oil that meets and/or exceeds at least two of the metrics can provide an economically viable biological oil for a biofuel. Meeting and/or exceeding more (additional) metrics can lead to a more robust process with greater likelihood of economic success. The different metrics can be somewhat competing with each other, such that optimization of one results in a lower outcome of another. However, the processes of the invention have been able to meet and/or exceed 2, 3, 4, 5, 6, and/or 7 of the metrics at least substantially simultaneously (including used in the same overall process) in any combination.

Metric refers to a standard of measurement and/or a key performance indicator, such as a measure of effectiveness, utilization, conversion, production, success, and/or the like.

Meeting refers to reaching, obtaining, satisfying, equaling, and/or the like.

Exceeding refers to extending beyond, to surpassing, and/or the like. According to some embodiments, exceeding includes at least 2 percent above threshold amount and/or quantity.

Metric A, a cell density (of the organism) measured in grams per liter (of the fermentation media or broth), measures and/or indicates productivity of the organism, utilization of the fermentation media (broth), and/or utilization of fermentation vessel volume. Increased cell density can result in increased product yields and increased utilization of equipment (lower capital costs). Generally, increased cell density is beneficial, but too high a cell density can result in higher pumping costs (increased viscosity) and/or difficulties in removing heat (lower heat transfer coefficient), and/or the like.

Density refers to a mass per unit volume of a material and/or a substance. Cell density refers to a mass of cells per unit volume, such as the weight of living cells per unit volume. It is commonly expressed as grams of dry cells per liter. The cell density can be measured at any suitable point in the method, such as upon commencing fermentation, during fermentation, upon completion of fermentation, over the entire batch, and/or the like.

The cell density metric can include any suitable value, such as at least about 50 grams dry weight per liter, at least about 100 dry weight grams per liter, at least about 115 dry weight grams per liter, at least about 125 dry weight grams per liter, at least about 150 grams dry weight per liter, at least about 175 grams dry weight per liter, at least about 200 grams dry weight per liter, at least about 250 grams dry weight per liter, at least about 350 grams dry weight per liter, less than about 400 grams dry weight per liter, 50 grams dry weight per liter to 350 grams dry weight per liter, 115 grams dry weight per liter to 200 grams dry weight per liter, and/or the like.

Metric B, a fatty acid content measured in percent on a dry mass basis, measures and/or indicates how much fatty acids, on a weight basis, are contained within the organism. Generally, a higher fatty acid content is desired and can provide for easier extraction and/or removal of the fatty acids from a remainder and/or residue of cellular material, as well as increased utilization and/or productivity for the feedstock and/or equipment.

Content refers to an amount of specified material contained. Dry mass basis refers to being at least substantially free from water. The fatty acid content can be measured at any suitable point in the method, such as upon commencing fermentation, during fermentation, upon completion of fermentation, over the entire batch, and/or the like.

The fatty acid content metric can include any suitable value, such as at least about 25 percent fatty acids on a dry mass basis, at least about 49 percent fatty acids on a dry mass basis, at least about 55 percent fatty acids on a dry mass basis, at least about 60 percent fatty acids on a dry mass basis, at least about 70 percent fatty acids on a dry mass basis, at least about 80 percent fatty acids on a dry mass basis, at least about 90 percent fatty acids on a dry mass basis, less than about 100 percent fatty acids on a dry mass basis, about 25 percent fatty acids on a dry mass basis to about 90 percent fatty acids on a dry mass basis, about 49 percent fatty acids on a dry mass basis to about 70 percent fatty acids on a dry mass basis, and/or the like.

Metric C, a fatty acid productivity measured in grams of fatty acids per liter of broth and/or fermentor volume per day (24 hours) of elapsed fermentation time measures and/or indicates a conversion rate and/or speed at which fatty acids are produced. Generally, a higher fatty acid productivity results in a more economic process since making product more rapidly (i.e., reduced cycle times) is desired.

Productivity refers to a quality and/or state of producing and/or making, such as a rate per unit of volume. The fatty acid productivity can be measured at any suitable point in the method, such as upon commencing fermentation, during fermentation, upon completion of fermentation, over the entire batch, and/or the like. The productivity can be measured on a fixed time, such as noon to noon each day. In the alternative, the productivity can be measured on a suitable rolling basis, such as for any 24 period. Other bases for measuring productivity are with the scope of the invention.

The fatty acid productivity metric can include any suitable value, such as at least about 5 grams of fatty acids per liter per day, at least about 10 grams fatty acids per liter per day, at least about 15 grams of fatty acids per liter per day, at least about 20 grams of fatty acids per liter per day, at least about 25 grams of fatty acids per liter per day, at least about 30 grams of fatty acids per liter per day, at least about 40 grams of fatty acids per liter per day, at least about 50 grams of fatty acids per liter per day, at least about 60 grams per liter per day, at least about 70 grams of fatty acids per liter per day, at least about 80 grams of fatty acids per liter per day, at least about 90 grams fatty acids per liter per day, less than about 100 grams fatty acids per liter per day, about 5 grams of fatty acids per liter per day to about 90 grams of fatty acids per liter per day, about 10 grams of fatty acids per liter per day to about 70 grams of fatty acids per liter per day, about 15 grams of fatty acids per liter per day to about 90 grams of fatty acids per liter per day and/or the like.

Metric D, a fatty acid yield measured in grams of fatty acid produced per grams of the feedstock consumed, measures and/or indicates a selectivity of the conversion of the feedstock into product. A higher fatty acid yield is generally preferred as it indicates carbon conversion from the sugar into fatty acid and not byproducts and/or cell mass.

Yield refers to an amount and/or quantity produced and/or returned. The fatty acid yield can be measured at any suitable point in the method, such as upon commencing fermentation, during fermentation, upon completion of fermentation, over the entire batch, and/or the like.

The fatty acid yield metric can include any suitable value, such as at least about 0.1 grams of fatty acids produced per grams of feedstock consumed, at least about 0.15 grams of fatty acids produced per grams of feedstock consumed, at least about 0.175 grams of fatty acids produced per grams of feedstock consumed, at least about 0.2 grams of fatty acids produced per grams of feedstock consumed, at least about 0.225 grams of fatty acids produced per grams of feedstock consumed, at least about 0.25 grams of fatty acids produced per grams of feedstock consumed; at least about 0.3 grams of fatty acids produced per grams of feedstock consumed, about 0.1 grams of fatty acids produced per grams of feedstock consumed to about 0.2 grams of fatty acids produced per grams of feedstock consumed, about 0.2 grams of fatty acids produced per grams of feedstock consumed and about 0.35 grams of fatty acids produced per grams of feedstock consumed, about 0.175 grams of fatty acids produced per grams of feedstock consumed to about 0.225 grams of fatty acids produced per grams of feedstock consumed, and/or the like.

Metric E, a 24 hour peak fatty acid productivity measured in grams of fatty acids per liter per day, measures and/or indicates an amount and/or rate of fatty acid produced over a 24 hour period.

The peak fatty acid productivity can include any suitable value, such as at least about 15 grams of fatty acids per liter per day, at least about 20 grams of fatty acids per liter per day, at least about 30 grams of fatty acids per liter per day, at least about 40 grams of fatty acids per liter per day, at least about 50 grams of fatty acids per liter per day, at least about 75 grams of fatty acids per liter per day, at least about 100 grams of fatty acids per liter per day, at least about 125 grams of fatty acids per liter per day, less than about 150 grams of fatty acids per liter per day, about 15 grams of fatty acids per liter per day to about 125 grams of fatty acids per liter per day, about 30 grams of fatty acids per liter per day to about 125 grams of fatty acids per liter per day, about 40 grams of fatty acids per liter per day to about 100 grams of fatty acids per liter per day, and/or the like.

Metric F, an extraction efficiency on a percent of total fatty acid content basis measures and/or indicates an amount of recovery of fatty acids available as product versus the total fatty acids produced by the organism.

The extraction efficiency can include any suitable value, such as at least about 25 percent of total fatty acid content, at least about 35 percent of total fatty acid content, at least about 45 percent of total fatty acid content, at least about 55 percent total fatty acid content, at least about 65 percent of total fatty acid content, at least about 75 percent of total fatty acid content, at least about 85 percent of total fatty acid content, at least about 95 percent of total fatty acid content, less than about 100 percent of total fatty acid content, about 25 percent of total fatty acid content to about 95 percent of total fatty acid content, about 25 percent of total fatty acid content to about 95 percent of total fatty acid content, about 35 percent of total fatty acid content to about 75 percent of total fatty acid content, and/or the like.

The extraction efficiency can be based on an extraction volume of any suitable value, such as at least about 1 liter, at least about 10 liters, at least about 100 liters, at least about 1,000 liters, less than about 10,000 liters, about 1 liter to about 10,000 liters, about 1 liter to about 1,000 liters, about 1 liter to about 100 liters, about 1 liter to about 10 liters, and/or the like. According to some embodiments, the volume for the extraction efficiency can be based on a volume of fermentation broth.

Metric G, a yield of fatty acids on oxygen expressed as grams of fatty acids produced per gram of oxygen consumed basis measures and/or indicates an amount and/or rate of oxygen used to produce the fatty acids. A higher oxygen demand can increase capital expenses and/or operating expenses.

The yield of fatty acid on oxygen can include any suitable value, such as more than about 0.4 grams of fatty acids produced per gram of oxygen consumed, more than about 0.5 grams of fatty acids produced per gram of oxygen consumed, more than about 0.6 grams of fatty acids produced per gram of oxygen consumed, more than about 0.7 grams of fatty acids produced per gram of oxygen consumed, more than about 0.8 grams of fatty acids produced per gram of oxygen consumed, more than about 0.9, less than about 1.0 grams of fatty acids produced per gram of oxygen consumed, about 0.4 grams of fatty acids produced per gram of oxygen consumed to about 0.9 grams of fatty acids produced per gram of oxygen consumed, about 0.6 grams of fatty acids produced per gram of oxygen consumed to about 0.8 grams of fatty acids produced per gram of oxygen consumed, and/or the like.

According to some embodiments, the method can include meeting or exceeding at least two or more of the metrics at least generally simultaneously or during production.

According to some embodiments, the method can include meeting or exceeding at least three or more of the metrics at least generally simultaneously or during production.

According to some embodiments, the method can include meeting or exceeding at least four or more of the metrics at least generally simultaneously or during production.

According to some embodiments, the method can include meeting or exceeding at least five or more of the metrics at least generally simultaneously or during production.

According to some embodiments, the method can include meeting or exceeding at least six or more of the metrics at least generally simultaneously or during production.

According to some embodiments, the method can include meeting or exceeding at least seven of the metrics at least generally simultaneously or during production.

The method can further include any suitable additional actions, such as extracting and/or removing the lipid containing fatty acids by cell lysing, applying pressure, solvent extraction, distillation, centrifugation, other mechanical processing, other thermal processing, other chemical processing, and/or the like. In the alternative, the producing organism can excrete and/or discharge the lipid containing fatty acids from the organism without additional processing.

The fatty acids can have any suitable profile and/or characteristics, such as generally suitable for biofuel production. According to some embodiments, the fatty acids can include a suitable amount and/or percent fatty acids with four or more double bonds on a mass basis. In the alternative, the fatty acids can include a suitable amount and/or percent fatty acids with three or more double bonds, with two or more double bonds, with one or more double bonds, and/or the like.

The suitable amount of double bonds can be less than about 25 percent weight percent of total fatty acids, less than about 15 percent weight percent of total fatty acids, less than about 10 percent weight percent of total fatty acids, less than about 5 percent weight percent of total fatty acids, less than about 3 percent weight percent of total fatty acids, less than about 2 percent weight percent of total fatty acids, less than about 1 percent weight percent of total fatty acids, less than about 0.5 percent weight percent of total fatty acids, less than about 0.1 percent weight percent of total fatty acids, at least about 5 percent weight percent of total fatty acids, about 25 percent weight percent of total fatty acids to about 0.1 percent weight percent of total fatty acids, about 10 percent weight percent of total fatty acids to about 5 percent weight percent of total fatty acids, and/or the like.

Fatty acids refer to saturated and/or unsaturated monocarboxylic acids, such as in the form of glycerides in fats and fatty oils. Glycerides can include acylglycerides, monoglycerides, diglycerides, triglycerides, and/or the like.

Double bonds refer two pairs of electrons shared by two atoms in a molecule.

In addition and/or the alternative, the resulting fatty acids can include at least about 30 percent monounsaturated fatty acids as weight percent of total fatty acids, at least about 40 percent monounsaturated fatty acids as weight percent of total fatty acids, at least about 50 percent monounsaturated fatty acids as weight percent of total fatty acids, at least about 60 percent monounsaturated fatty acids as weight percent total fatty acids, at least about 70 percent monounsaturated fatty acids as weight percent of total fatty acids, at least about 80 percent monounsaturated fatty acids as weight percent of total fatty acids, at least about 90 percent monounsaturated fatty acids as weight percent of total fatty acids, about 30 percent monounsaturated fatty acids as weight percent of total fatty acids to about 90 percent monounsaturated fatty acids as weight percent of total fatty acids, less than about 100 percent monounsaturated fatty acids as weight percent of total fatty acids, about 50 percent monounsaturated fatty acids as weight percent of total fatty acids to about 70 percent monounsaturated fatty acids as weight percent of total fatty acids, and/or the like. Monounsaturated refers to molecules having one double bond.

According to some embodiments, the lipid can include any suitable amount and/or percent of saturated fatty acids on a total fatty acid mass basis. The suitable amount and/or percent of saturated fatty acids can include less than about 5 percent of total fatty acids as weight percent total fatty acids, less than about 10 percent of total fatty acids as weight percent total fatty acids, less than about 20 percent of total fatty acids as weight percent total fatty acids, less than about 25 percent of total fatty acids as weight percent total fatty acids, less than about 30 percent of total fatty acids as weight percent total fatty acids, less than about 35 percent of total fatty acids as weight percent total fatty acids, less than about 40 percent of total fatty acids as weight percent total fatty acids, less than about 50 percent of total fatty acids as weight percent total fatty acids, less than about 60 percent of total fatty acids as weight percent total fatty acids, at least about 1 percent of total fatty acids as weight percent total fatty acids, about 10 percent of total fatty acids as weight percent total fatty acids to about 60 percent of total fatty acids as weight percent total fatty acids, about 25 percent of total fatty acids as weight percent total fatty acids to about 40 percent of total fatty acids as weight percent total fatty acids, about 1 percent of total fatty acids as weight percent total fatty acids to about 10 percent of total fatty acids as weight percent total fatty acids and/or the like. Saturated refers to compounds with no double bonds and/or triple bonds between atoms (carbon atoms).

The biological oil can be further processed into the biofuel with any suitable method, such as esterification, transesterification, hydrogenation, cracking, and/or the like. In the alternative, the biological oil can be suitable for direct use as a biofuel. Esterification refers to making and/or forming an ester, such as by reacting an acid with an alcohol to form an ester. Transesterification refers to changing one ester into one or more different esters, such as by reaction of an alcohol with a triglyceride to form fatty acid esters and glycerol, for example. Hydrogenation and/or hydrotreating refer to reactions to add hydrogen to molecules, such as to saturate and/or reduce materials.

In addition and/or the alternative, the resulting fatty acids upon transesterification can have any suitable fatty acid methyl ester profile. The profile (expressed as weight percent of total fatty acids) can include about 30 percent oleic acid to about 90 percent oleic acid, about 50 percent oleic acid to about 70 percent oleic acid, about 60 percent oleic acid, and/or the like, all on a mass basis. The profile can include about 10 percent linoleic acid to about 70 percent linoleic acid, about 30 percent linoleic acid to about 50 percent linoleic acid, about 15 percent linoleic acid to about 35 percent linoleic acid, about 40 percent linoleic acid, and/or the like.

Transesterification can include use of any suitable alcohol, such as methanol, ethanol, propanol, butanol, and/or the like.

According to some embodiments, the fatty acid methyl ester profile (expressed as weight percent of total fatty acids) can include: about 1 percent palmitic acid to about 10 percent palmitic acid; about 0.5 percent stearic acid to about 2.5 percent stearic acid; about 50 percent oleic acid to about 70 percent oleic acid; about 15 percent linoleic acid to about 35 percent linoleic acid; and/or about 6 percent linolenic acid to about 12 percent linolenic acid.

According to some embodiments, the fatty acid methyl ester profile (expressed as weight percent of total fatty acids) can include: about 0 percent myristic acid to about 1.5 percent myristic acid; about 1 percent palmitic acid to about 10 percent palmitic acid; about 0.5 percent stearic acid to about 2.5 percent stearic acid; about 0 percent arachidic acid to about 1.5 percent arachidic acid; about 0 percent behenic acid to about 1.5 percent behenic acid; about 0 percent lignoceric acid to about 2 percent lignoceric acid; about 0 percent palmitoleic acid to about 1 percent palmitoleic acid; about 50 percent oleic acid to about 70 percent oleic acid, about 0 percent eicosenoic acid to about 3 percent eicosenoic acid; about 0 percent erucic acid to about 5 percent erucic acid, about 15 percent linoleic acid to about 35 percent linoleic acid; and/or about 6 percent linolenic acid to about 12 percent linolenic acid.

Other embodiments of different fatty acid ester profiles are within the scope of the invention.

The resulting biofuel can meet and/or exceed international standards EN 14214:2008 (Automotive fuels, Fatty acid methyl esters (FAME) for diesel engines) and/or ASTM D6751-09 (Standard Specification for Biodiesel Fuel Blend Stock (B100) for Middle Distillate Fuels). The entire contents of EN 14214:2008 and ASTM D6751-09 are hereby both incorporated by reference in their entirety as a part of this specification.

According to some embodiments, the fatty acids can include a profile at least substantially similar to the fatty acids found in rapeseed. Substantially refers to being largely that which is specified and/or identified. Similar refers to having characteristics in common, such as not dramatically different. Substantially similar can include having a profile at least about 50 percent like rapeseed, at least about 60 percent like rapeseed, at least about 70 percent like rapeseed, at least about 80 percent like rapeseed, at least about 90 percent like rapeseed, at least about 95 percent like rapeseed, at least 99 percent like rapeseed, less than about 90 percent like rapeseed, about 50 percent like rapeseed to about 99 percent like rapeseed, and/or the like, all on a weight percent total fatty acid basis, as measured, for example, by correlation analysis.

Producing cell mass and/or producing lipid containing fatty acids can occur at any suitable condition, such as a temperature of at least about 18 degrees Celsius, at least about 20 degrees Celsius, at least about 25 degrees Celsius, at least about 30 degrees Celsius, at least about 35 degrees Celsius, at least about 40 degrees Celsius, at least about 45 degrees Celsius, at least about 50 degrees Celsius, less than about 100 degrees Celsius, about 18 degrees Celsius to about 50 degrees Celsius, about 20 degrees Celsius to about 30 degrees Celsius, and/or the like. Operating at high temperatures can increase yield, increase productivity, reduce growth of foreign organisms, and/or the like. Consuming and/or producing can occur at a same temperature and/or a different temperature. Embodiments with temperature transients (change in temperature with respect to time) during the consuming and/or producing are within the scope of the invention.

According to some embodiments, the method and/or process can include temperature control, such as by addition of heat, cooling, and/or the like. Heat can be supplied by steam, saturated stream, super heated stream, hot water, glycol, heat transfer oil, heat transfer fluid, other process streams, and/or the like. Cooling can be supplied by cooling water, refrigerant, brine, glycol, heat transfer fluid, coolant, other process streams, and/or the like. Temperature control can use any suitable technique and/or configuration, such as indirect heat exchange, direct heat exchange, convection, conduction, radiation, and/or the like.

According to some embodiments, the method can include extracting the fatty acids from the organism, where the extracting has an efficiency of at least about 70 percent, at least about 75 percent, at least about 80 percent, at least about 85 percent, at least about 90 percent, at least about 95 percent, less than about 100 percent, about 70 percent to about 95 percent, about 80 percent to about 95 percent, and/or the like, all on a percent of total fatty acid content basis.

Extracting can include solvent extraction with ethanol, hexane, other alcohols, other suitable solvents (chemical solvents, physical solvents, non-polar solvents, polar solvents, and/or supercritical solvents), and/or the like.

According to some embodiments, the fatty acid productivity of the organism meets and/or exceeds at least about 30 grams per liter per day, and/or the fatty acid yield meets and/or exceeds at least about 0.175 grams of fatty acids produced per grams of carbon in the feedstock.

According to some embodiments, the 24 hour peak fatty acid productivity meets and/or exceeds at least about 50 grams per liter per day.

According to some embodiments, a 6 hour peak fatty acid productivity meets and/or exceeds at least about 70 grams per liter per day.

According to some embodiments, consuming and/or producing occur under nitrogen limitation. Nitrogen limitation refers to lacking in nitrogen, such as used in cellular reproduction. In the alternative, at least a portion of consuming and/or producing occur with nitrogen addition, such as from ammonia.

According to some embodiments, producing an organism and/or producing a lipid containing fatty acids occurs independent of sugar fermentation, such as in a separate vessel. In the alternative, producing an organism and/or producing a lipid containing fatty acids occurs at least somewhat contemporaneously and/or simultaneously with sugar fermentation, such as in a same vessel.

Consuming and/or producing can occur at any suitable pH, such as a pH of below about 3, a pH of below about 5, a pH of below about 6, a pH of about 7.0 or below, a pH of about 7, a pH of at least about 8, a pH of at least about 9, a pH of at least about 10, a pH of about 5 to about 9, a pH of about 6 to about 8, a pH of about 7 to about 8, and/or the like. Operating at different pH levels can inhibit growth of foreign organisms. Inhibiting growth of foreign organisms can allow for operation of the method to occur without a need for a separate sterilization process, such as at beginning of each batch. Embodiments with changes in pH during operation are within the scope of the invention.

Sterilization can consume energy, time, and/or other resources. Therefore, in some embodiments, a sterilization process is not utilized, for example when the pH level inhibits growth of foreign organisms, as discussed above. In the alternative, the method can further include a sterilization process, such as steaming to at least a threshold temperature for a suitable duration.

According to some embodiments, the invention can include a process of reducing energy costs and/or capital costs of a fermentation process for producing the oil where growth of the organism occurs at a temperature that is optimal for growth, while a lipid containing fatty acid production phase occurs at a temperature at least about 2 degrees Celsius, at least about 3 degrees Celsius, at least about 4 degrees Celsius, at least about 5 degrees Celsius, at least about 7 degrees Celsius, less than about 20 degrees Celsius, about 2 degrees Celsius to about 7 degrees Celsius, about 4 degrees Celsius to about 5 degrees Celsius, and/or the like higher than the optimal growth temperature.

According to some embodiments, the invention can include a biological oil made by any of the methods, units, and/or organisms disclosed within this specification.

According to some embodiments, the invention can include a biofuel made from any of the biological oils disclosed within this specification.

According to some embodiments, the invention can include a biofuel suitable for use in compression engines. The biofuel can include a fatty acid methyl ester profile. The fatty acid methyl ester profile can include from about 50 percent to about 70 percent oleic acid on a weight percent of total fatty acids basis, and/or from about 15 percent to about 35 percent linolenic acid on a on a weight percent of total fatty acids basis.

According to some embodiments, the fatty acid methyl ester profile derives from fatty acids produced by an organism from kingdom stramenopile, kingdom fungi, and/or the like.

According to some embodiments, the invention can include a unit for producing biological oil. The unit can include a feedstock stream, a vessel connected to the feedstock stream, an organism disposed within the vessel, and/or a lipid stream connected to the vessel. The organism can meet and/or exceed at least two metrics, where the metrics include: A) a cell density of at least about 115 grams per liter; B) a fatty acid content of at least about 49 percent on a dry mass basis; C) a fatty acid productivity of at least about 15 grams per liter per day; D) a fatty acid yield of at least about 0.175 grams of fatty acids per grams of the feedstock consumed; E) a 24 hour peak fatty acid productivity of at least about 30 grams per liter per day; F) an extraction efficiency of at least about 65 percent on a percent of total fatty acid content basis; and/or G) a yield of fatty acids on oxygen expressed as grams of fatty acids produced per gram of oxygen consumed of at least about 0.4.

Unit refers to a single quantity regarded as a whole, a piece and/or complex of apparatus serving to perform one or more particular functions and/or outcomes, and/or the like.

Stream refers to a flow and/or a supply of a substance and/or a material, such as a steady succession. Flow of streams can be continuous, discrete, intermittent, batch, semibatch, semicontinuous, and/or the like.

Vessel refers to a container and/or holder of a substance, such as a liquid, a gas, a fermentation broth, and/or the like. Vessels can include any suitable size and/or shape, such as at least about 1 liter, at least about 1,000 liters, at least about 100,000 liters, at least about 1,000,000 liters, at least about 1,000,000,000 liters, less than about 1,000,000 liters, about 1 liter to about 1,000,000,000 liters, and/or the like. Vessels can include tanks, reactors, columns, vats, barrels, basins, and/or the like. Vessels can include any suitable auxiliary equipment, such as pumps, agitators, aeration equipment, heat exchangers, coils, jackets, pressurization systems (positive pressure and/or vacuum), control systems, and/or the like.

Dispose refers to put in place, to put in location, to set in readiness, and/or the like. The organism can be freely incorporated into a fermentation broth (suspended), and/or fixed upon a suitable media and/or surface within at least a portion of the vessel. The organism can be generally denser than the broth (sink), generally lighter than the broth (float), generally neutrally buoyant with respect to the broth, and/or the like.

Adapted refers to make fit for a specific use, purpose, and/or the like.

According to some embodiments, the organism disposed within the vessel can include an organism from kingdom stramenopile, kingdom fungi, and/or the like.

According to some embodiments, the vessel operates on a batch basis, a discrete basis, a semibatch basis, a semicontinuous basis, a continuous basis, and/or the like. Combinations of series and/or parallel vessels are within the scope of the invention.

According to some embodiments, the invention can include an isolated organism for producing a biological oil in which one or more of the following overall indices for fatty acid efficiency are achieved, where OILE, OILE1, OILE2, OILE3 and OILE4 can be defined as:

OILE=A*B*C*D*E*F*G

OILE1=C*D*F

OILE2=A*B*C*D*F

OILE3=D*E*F

OILE4=A*B*C*D*F*G

Where:
A=a cell density in grams per liter;
B=a fatty acid content on a dry percent mass basis;
C=a fatty acid productivity in grams per liter per day;
D=a fatty acid yield in grams of fatty acids per grams of feedstock consumed;
E=a 24 hour peak fatty acid productivity in grams per liter per day;
F=an extraction efficiency on a percent fatty acid content basis; and/or
G=a yield of fatty acid on oxygen expressed on a grams of fatty acids produced per gram of oxygen consumed basis.

Metrics B and F are measured as percentages. When calculating the values for OILE, OILE1, OILE2, OILE3, and/or OILE4, the percentages for B and F are entered as a decimal (less than 1.0).

According to some embodiments, the invention can include a method of isolating an organism for producing a biological oil including identifying an organism wherein one or more of the overall indices discussed above (OILE, OILE1, OILE2, OILE3, and OILE4) for fatty acid efficiency are achieved and then isolating the organism.

According to some embodiments, the isolated organism can include an overall index for fatty acid efficiency (OILE) of any suitable value, such as at least about 811, at least about 4,958, at least about 20,292, at least about 33,342, at least about 53,000, about 811 to about 53,000, about 4,958 to about 53,000, about 4,958 to about 20,292, about 4,958 to about 33,342, about 20,292 to about 53,000, about 20,292 to about 33,342, about 33,342 to about 53,000, and/or the like.

According to some embodiments, the isolated organism can include an overall index for fatty acid efficiency one (OILE1) of any suitable value, such as at least about 1.5, at least about 2.9, at least about 5.1, at least about 6.3, at least about 7.6, about 1.5 to about 7.6, about 2.9 to about 7.6, about 2.9 to about 6.3, about 2.9 to about 5.1, about 5.1 to about 7.6, about 5.1 to about 6.3, about 6.3 to about 7.6, and/or the like.

According to some embodiments, the isolated organism can include an overall index for fatty acid efficiency two (OILE2) of any suitable value, such as at least about 73, at least about 235, at least about 593, at least about 803, at least about 1,000, about 73 to about 1,000, about 235 to about 1,000, about 235 to about 803, about 235 to about 593, about 593 to about 1,000, about 593 to about 803, about 803 to about 1,000, and/or the like.

According to some embodiments, the isolated organism can include an overall index for fatty acid efficiency three (OILE3) of any suitable value, such as at least about 2.2, at least about 4.4, at least about 7.6, at least about 9.4, at least about 11.4, about 2.2 to about 11.4, about 4.4 to about 11.4, about 4.4 to about 9.4, about 4.4 to about 7.6, about 7.6 to about 11.4, about 7.6 to about 9.4, about 9.4 to about 11.4, and/or the like.

According to some embodiments, the isolated organism can include an overall index for fatty acid efficiency four (OILE4) of any suitable value, such as at least about 32, at least about 142, at least about 457, at least about 682, at least about 990, about 32 to about 990, about 142 to about 990, about 142 to about 682, about 142 to about 457, about 457 to about 990, about 457 to about 682, about 682 to about 990 and/or the like.

According to some embodiments, the isolated organism for OILE, OILE1, OILE2, OILE3 or OILE4 can include naturally occurring organisms, genetically modified organisms, and/or the like.

According to some embodiments, any of the methods, units, and/or organisms disclosed within this specification can use an organism that consumes a carbon feedstock where the carbon feedstock includes at least about 1 percent, at least about 5 percent, at least about 10 percent, at least about 15 percent, at least about 20 percent, at least about 25 percent, about 1 percent to about 25 percent and/or the like of total nitrogen consumed, all on a mass basis.

### EXAMPLES

### Example 1

About 1,500 strains of microorganisms were tested for nonphotosynthetic fatty acid production capabilities. Strains refer to a group and/or collection of presumed common ancestry, such as with clear cut physiological distinctions but not necessarily morphological distinctions. The strains of tested organisms included microalgae, yeast, and yeast like organisms. The tested organisms include naturally occurring isolated organisms as well as some genetically modified organisms. The tests examined fatty acid producing capabilities and potential biofuel characteristics based on fatty acid profiles. The organisms were screened based on meeting the key performance metrics discussed above. The primary criteria were a potential for good growth and high fat accumulation. The secondary criteria were fatty acid profiles being similar to rapeseed oil.

Typical ranges for fatty acid profiles (expressed as weight percent of total fatty acids) based on rapeseed oil include about 1 percent to about 10 percent of 16:0, about 0.5 percent to about 2.5 percent of 18:0, about 50 percent to about 70 percent 18:1, about 15 to about 35 percent 18:2, about 6 percent to about 12 percent 18:3, and less than about 1 percent polyunsaturated fatty acids, all on a mass basis. The first number in the fatty acid nomenclature represents a number of carbon atoms in a molecule and the second indicates a number of double bonds in the molecule.

The strains meeting the primary and secondary criteria produced at least about 8 grams per liter of dry biomass weight and had a fatty acid content of at least about 35 percent of dry weight when grown under the screening conditions in shake flasks. Applying these criteria narrowed the about 1,500 strains down to 154 strains (“the identified strains”) for additional testing. The identified strains had a dry biomass weight of 5.6 grams per liter to 27.3 grams per liter. The identified strains had a fatty acid content of 15 percent to 74 percent of dry weight. The identified strains produced a fatty acid profile (expressed as weight percent of total fatty acids) with 14:0 of 0 percent to 37 percent, 16:0 of 11 percent to 54 percent, 16:1 of 0 percent to 23 percent, 18:0 of 1 percent to 24 percent, 18:1 of 0 percent to 66 percent, 18:2 of 0 percent to 45 percent, 18:3 of 0 percent to 9 percent, and a polyunsaturated fatty acid content of 0 percent to 44 percent.

### Example 2

The 154 strains identified in Example 1 above were prepared for flask testing. The strains were tested for an ability to grow on several potential media. The tested media included glucose, sucrose, glycerol, fructose, and/or xylose. The media sources used pure sugars sources. For this experiment, the standard culture medium was modified to eliminate of organic carbons, such as yeast extract and monosodium glutamate. Eliminating organic carbons allows each strain to be tested for growth on the specified substrate.

The shake flasks were prepared with a medium for standard shake flask medium without the yeast extract. An inoculum of each strain was grown in standard shake flask medium including glucose. 0.5 milliliters of the inoculum was used to inoculate 49.5 milliliters of experimental medium. Six different types of media were used for each strain. One type of media corresponded to each type of sugar tested (glucose, sucrose, glycerol, fructose, xylose) and an additional medium that contained no added organic carbon as a control for any growth that occurred as a result of carryover of nutrients from the inoculum. Each strain was grown in each type of medium in shake flasks for seven (7) days at 22.5 degrees Celsius and 200 revolutions per minute. The contents of each flask were harvested by centrifugation, washed, freeze dried, and the biomass weights determined. For samples resulting in a biomass dry weight of greater than 1 gram per liter, a total fat content was determined by fatty acid methyl ester (FAME) analysis. Samples with a biomass weight of less than 1 gram per liter did not produce sufficient material for analysis.

### Example 3

Seven (7) of the yeast strains from Example 2 that grew on sucrose were utilized for preliminary extraction tests. Ten (10) milliliters of hexane were added to one (1) gram of freeze dried biomass in a Swedish tube with 3 stainless steel balls. The tube was shaken at high speed for 3 hours. The mixture was filtered using 25 micron filter paper. The biomeal was washed three times with an additional ten (10) milliliters of hexane each time and filtered each time to form a combined filtrate. The combined filtrate was evaporated, and recovered oil weighed. Extraction yield was calculated using esterified biomass and biomeal samples.

The same seven (7) strains were also evaluated using an aqueous extraction procedure. One (1) gram of freeze dried biomass was reconstituted with six (6) grams of water to make a solution with about 14 percent biomass. This solution was processed using standard extraction methods. Extraction yields using hexane as the solvent ranged from 48 percent to 94 percent for different strains. Schizochytrium had an extraction yield of 86 percent. Some strains can have stronger than expected cell walls and can benefit from additional passes to effectively break the cells. There was no significant oil layer from the standard extraction procedure. There may have been a few small oil droplets present for some strains, but yields were still relatively low. Modifications to extraction procedures for the yeast strains man improve yields.

### Sucrose

Twenty-six (26) strains that produce on sucrose media were analyzed and had a dry biomass weight of 1.3 grams per liter to 3.1 grams per liter. The identified strains had a fatty acid content of 36 percent to 73 percent of dry weight. The identified strains produced a fatty acid profile (as weight percent total fatty acids) with 14:0 of 0 percent to 17 percent, 16:0 of 13 percent to 33 percent, 16:1 of 0 percent to 13 percent, 18:0 of 1 percent to 24 percent, 18:1 of 3 percent to 59 percent, 18:2 of 0 percent to 29 percent, 18:3 of 0 percent to 9 percent, and a polyunsaturated fatty acid content of 0 percent to 31 percent.

Members of the genera Rhodotorula and Rhodosporidium were consistently among the better performers from the natural isolates and publicly available strains. Additionally, members of the genera Sporobolomyces and Spordiobolus also performed better than other genera. These four (4) genera were found to be oleaginous and suitable for production of biofuels. An additional two genera, Pseudozyma and Cryptococcus, were also found to be good performers. Specifically, the Pseudozyma had a dry biomass weight of 2 grams per liter and a fatty acid content of 57 percent of dry weight. The Pseudozyma produced a fatty acid profile (expressed as weight percent of total fatty acids) with 14:0 of 0 percent, 16:0 of 13 percent, 16:1 of 3 percent, 18:0 of 4 percent, 18:1 of 59 percent, 18:2 of 14 percent, 18:3 of 0 percent, and a polyunsaturated fatty acid content of 0 percent.

### Xylose

Twelve (12) strains that produce on xylose media were analyzed and had a dry biomass weight of 0.4 grams per liter to 2.9 grams per liter. The identified strains had a fatty acid content of 38 percent to 69 percent. The identified strains produced a fatty acid profile (expressed as weight percent of total fatty acids) with 14:0 of 0 percent to 1 percent, 16:0 of 12 percent to 32 percent, 16:1 of 0 percent to 11 percent, 18:0 of 2 percent to 52 percent, 18:1 of 14 percent to 50 percent, 18:2 of 10 percent to 32 percent, 18:3 of 0 percent to 7 percent, and a polyunsaturated fatty acid content of 0 percent to 2 percent.

Some of the strains analyzed in xylose media resulted in a limited number of strains with a high fatty acid accumulation. In general, these strains did not have fatty acid accumulations comparable to the other sugars. Only three (3) strains had greater than 50 percent fatty acid content. Yeasts tended to perform better than other strains as xylose isolates and most were also among the better performing strains on sucrose. Specifically, the Pseudozyma had a dry biomass weight of 2 grams per liter and a fatty acid content of 55 percent of dry weight. The Pseudozyma produced a fatty acid profile (expressed as weight percent of total fatty acids) with 14:0 of 1 percent, 16:0 of 16 percent, 16:1 of 4 percent, 18:0 of 5 percent, 18:1 of 50 percent, 18:2 of 16 percent, 18:3 of 0 percent, and a polyunsaturated fatty acid content of 0 percent.

### Glucose

Twenty-six (26) strains that produce on glucose media were analyzed and had a dry biomass weight of 2.0 grams per liter to 3.3 grams per liter. The identified strains had a fatty acid content of 50 percent to 80 percent of dry weight. The identified strains produced a fatty acid profile (expressed as weight percent of total fatty acids) with 14:0 of 0 percent to 26 percent, 16:0 of 18 percent to 42 percent, 16:1 of 0 percent to 25 percent, 18:0 of 0 percent to 26 percent, 18:1 of 5 percent to 54 percent, 18:2 of 0 percent to 26 percent, 18:3 of 0 percent to 6 percent, and a polyunsaturated fatty acid content of 0 percent to 23 percent.

Microalgae isolates, such as Schizochytrium, performed better in glucose media than other strains. Most of these strains had at least 60 percent fatty acid content levels and several with fatty acid contents above 70 percent. A number of strains did not grow well on glucose in the modified media, even though they did grow well in the original screening media containing monosodium glutamate and yeast extract. Specifically, the Schizochytrium had a dry biomass weight of 2.8 grams per liter and a fatty acid content of 67 percent of dry weight. The Schizochytrium produced a fatty acid profile (expressed as weight percent of total fatty acids) with 14:0 of 19 percent, 16:0 of 31 percent, 16:1 of 21 percent, 18:0 of 1 percent, 18:1 of 6 percent, 18:2 of 0 percent, 18:3 of 0 percent, and a polyunsaturated fatty acid content of 21 percent.

### Fructose

Twenty (20) strains that produce on fructose media were analyzed and had a dry biomass weight of 2.0 grams per liter to 3.4 grams per liter. The identified strains had a fat content of 37 percent to 81 percent of dry weight. The identified strains produced a fatty acid profile (expressed as weight percent of total fatty acids) with 14:0 of 0 percent to 27 percent, 16:0 of 17 percent to 45 percent, 16:1 of 0 percent to 17 percent, 18:0 of 0 percent to 24 percent, 18:1 of 5 percent to 46 percent, 18:2 of 0 percent to 30 percent, 18:3 of 0 percent to 7 percent, and a polyunsaturated fatty acid content of 0 percent to 23 percent.

Microalgae isolates, such as Schizochytrium, performed better in fructose media than other strains. Most of these strains had at least 60 percent fatty acids as a percent dry weight with several above 70 percent. There was considerable overlap between the top performers on glucose and fructose. Specifically, the Schizochytrium had a dry biomass weight of 2.7 grams per liter and a fatty acid content of 65 percent of dry weight. The Schizochytrium produced a fatty acid profile (expressed as weight percent of total fatty acids) with 14:0 of 20 percent, 16:0 of 32 percent, 16:1 of 17 percent, 18:0 of 1 percent, 18:1 of 6 percent, 18:2 of 0 percent, 18:3 of 0 percent, and a polyunsaturated fatty acid content of 23 percent.

### Glycerol

Twenty (20) strains that produce on glycerol media were analyzed and had a dry biomass weight of 2 grams per liter to 3.5 grams per liter. The identified strains had a fatty acid content of 38 percent to 74 percent of dry weight. The identified strains produced a fatty acid profile (expressed as weight percent of total fatty acids) with 14:0 of 1 percent to 22 percent, 16:0 of 17 percent to 43 percent, 16:1 of 0 percent to 23 percent, 18:0 of 0 percent to 26 percent, 18:1 of 6 percent to 44 percent, 18:2 of 0 percent to 16 percent, 18:3 of 0 percent to 5 percent, and a polyunsaturated fatty acid content of 0 percent to 28 percent.

Microalgae isolates, such as Schizochytrium, performed better in fructose media than other strains. Many of the strains had at least a 60 percent fatty acid content and there was also considerable overlap between the algal strains for both glucose and fructose. Specifically, the Schizochytrium had a dry biomass weight of 3.4 grams per liter and a fatty acid content of 68 percent of dry weight. The Schizochytrium produced a fatty acid profile (expressed as weight percent of total fatty acids) with 14:0 of 19 percent, 16:0 of 28 percent, 16:1 of 22 percent, 18:0 of 1 percent, 18:1 of 7 percent, 18:2 of 0 percent, 18:3 of 0 percent, and a polyunsaturated fatty acid content of 21 percent. Furthermore, the Pseudozyma had a dry biomass weight of 2 grams per liter and a fat content of 62 percent or 1.2 grams per liter. The Pseudozyma produced a fatty acid profile (expressed as weight percent of total fatty acids) with 14:0 of 1 percent, 16:0 of 21 percent, 16:1 of 5 percent, 18:0 of 7 percent, 18:1 of 44 percent, 18:2 of 15 percent, 18:3 of 0 percent, and a polyunsaturated fatty acid content of 0 percent.

A broad range of fatty acid profiles were observed in the strains selected for evaluation over the different media. The fatty acid profiles (expressed as weight percent of total fatty acids) ranged from greater than 50 percent to less than 1 percent polyunsaturated fatty acids and greater than 60 percent to less than 5 percent monounsaturated fatty acids. Generally, the microalgae had the broadest range of fatty acids, while the yeast were all relatively very low polyunsaturated fatty acids with relatively high monounsaturates. Generally, the yeast strains had a fatty acid profile that had the at least some similarity to the targeted rape seed profile.

With respect to public culture collection strains, several representative strains had been ordered from public culture collections for evaluation. Seven (7) of these strains grew reasonably well on glucose and fructose containing media. Lipomyces starkeyi failed to grow on two separate occasions. Two of these strains (Rhodotortula glutinis and Pseudozyma aphidis) had good growth and fatty acid accumulation on sucrose. None of the public strains had good growth or fatty acid contents on xylose.

### Example 4

The lead strains with good fatty acid production on sucrose from Example 3 were used for follow up flask experiments to better understand the fatty acid accumulation kinetics and their ability to produce fatty acids at lower salinity (chloride) conditions (since originally isolated from marine environments). Each strain was grown in shake flasks in shake flask medium at four (4) different levels of sodium chloride (NaCl) (25 grams per liter, 12.5, grams per liter, 0.625 grams per liter, and 0 grams per liter). All flasks were incubated at 22.5 degrees Celsius at 200 revolutions per minute of shaking. One flask from each salinity condition for each strain was harvested daily and the dry biomass weight and fatty acid content were determined.

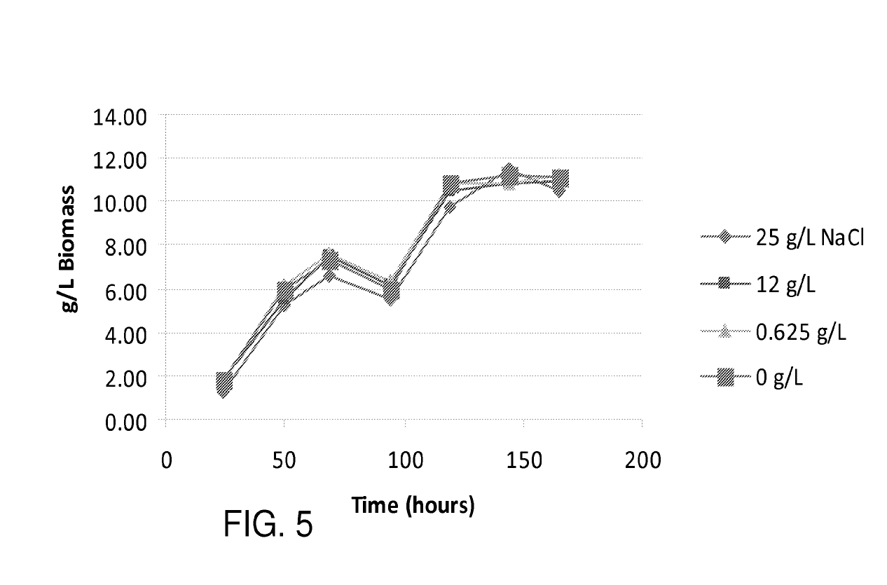

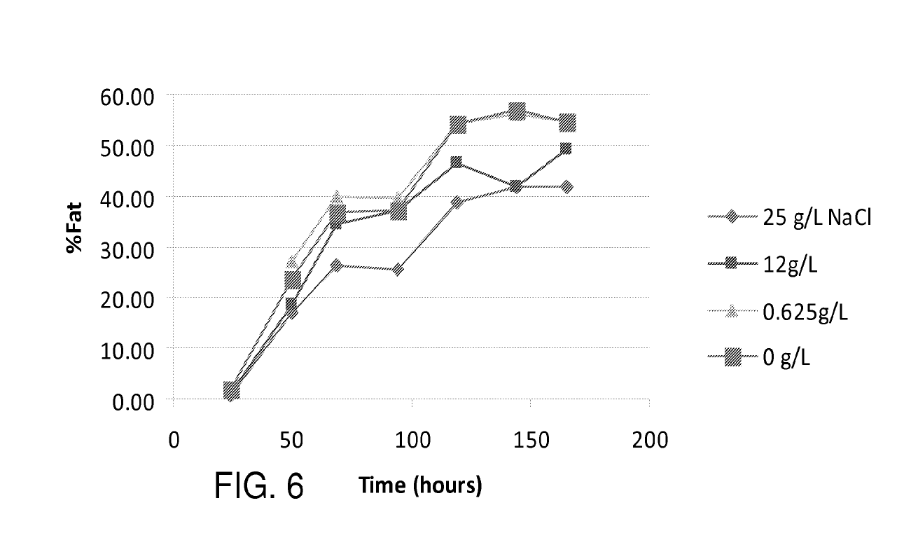

FIGS. 5 and 6 show biomass and percent fat for Pseudozyma for different sodium chloride levels with respect to time.

All six (6) of these strains generally grew as well or better at a lower chloride level (0 grams per liter or 0.625 grams per liter of NaCl) as they did at a normal NaCl level of 25 grams per liter. At the same time, all strains accumulated a significant amount of fatty acids under the low chloride conditions. This data suggests that reductions in salinity level for the yeast strains should not have a negative impact on the fatty acid accumulation and could in fact improve productivity.

### Example 5

In this example, the thraustochytrid Schizochytrium sp. (ATCC 20888) was cultivated in a 100 liter New Brunswick Scientific (Edison, N.J., U.S.A.) BioFlo 6000 fermentor with a carbon (glucose) and nitrogen (ammonium hydroxide) fed-batch process. The fermentation was inoculated with 6 liters of culture. For inoculum propagation a 14 liter VirTis (SP Scientific Gardiner, New York, U.S.A.) fermentor was utilized. The inoculum medium included 10 liters of medium prepared in four separate groups. Group A included 98 grams MSG*1H₂O, 202 grams Na₂SO₄, 5 grams KCl, 22.5 grams MgSO₄*7H₂O, 23.1 grams (NH₄)₂SO₄, 14.7 grams KH₂O₄, 0.9 grams CaCl₂*2H₂O, 17.7 milligrams MnCl₂*4H₂O, 18.1 milligrams ZnSO₄*7H₂O, 0.2 milligrams CoCl₂*6H₂O, 0.2 milligrams Na₂MoO₄*2H₂O, 11.8 milligrams CuSO₄*5H₂O, 11.8 milligrams NiSO₄*6H₂O, and 2 milliliters Dow (Midland, Mich., U.S.A.) 1520US (antifoam). Group A was autoclaved at 121 degrees Celsius in the inoculum fermentor at a volume of approximately 9.5 liters. Group B included 20 milliliters of a one liter stock solution containing 2.94 grams FeSO₄*7H₂O and 1 grams citric acid. The group B stock solution was autoclaved at 121 degrees Celsius. Group C included 37.6 milligrams thiamine-HCl, 1.9 milligrams vitamin B12, and 1.9 milligrams pantothenic acid hemi-calcium salt dissolved in 10 milliliters and filter sterilized. Group D included 1,000 milliliters of distilled water containing 400 grams glucose powder. After the fermentor was cooled to 29.5 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation.

The inoculum fermentor was inoculated with 18 milliliters of a standard ATCC 20888 shake flask culture and cultivated at 29.5 degrees Celsius, pH 5.5, 350 revolutions per minute agitation, and 8 liters per minute of air for a period of 27 hours, at which point 6 liters of inoculum broth were transferred to the 100 liter fermentor. The 100 liter fermentor included 80 liters of fermentation media. The fermentation media was prepared in a similar fashion to the inoculum fermentor. The fermentation media included 7 batched media groups. Group A included 1,089.6 grams Na₂SO₄, 57.6 grams K₂SO₄, 44.8 grams KCl, 181.6 grams MgSO₄*7H₂O, and 90.4 grams KH₂PO₄. Group A was steam sterilized at 122 degrees Celsius for 60 minutes in the 100 liter fermentor at a volume of approximately 35 liters. Group B included 90.4 grams (NH₄)₂SO₄ and 10.4 grams MSG*1H₂O in a volume of approximately 500 milliliters. Group C included 15.2 grams CaCl₂*2H₂O in a volume of approximately 200 milliliters. Group D included 1,200 grams of powdered glucose in approximately 2 liters of distilled water. Group E included 248 milligrams MnCl₂*4H2O, 248 milligrams ZnSO₄*7H₂O, 3.2 milligrams CoCl₂*6H₂O, 3.2 milligrams Na₂MoO₄*2H₂O, 165.6 milligrams CuSO₄*5H₂O, and 165.6 milligrams NiSO₄*6H₂O in a volume of approximately 1 liter. Group F included 824 milligrams FeSO₄*7H₂O and 280.3 milligrams citric acid in a volume of approximately 280 milliliters. Group G included 780 milligrams thiamine-HCl, 12.8 milligrams vitamin B12, and 266.4 milligrams pantothenic acid hemi-calcium salt filter sterilized in a volume of approximately of 67.4 milliliters distilled water. Groups B, C, D, E, F, and G were combined and added to the fermentor after the fermentor reached an operating temperature of 29.5 degrees Celsius. The fermentor volume prior to inoculation was approximately 38 liters.

The fermentor was inoculated with 6 liters of broth from the fermentation described above. The fermentation was pH controlled utilizing a 5.4 liter solution of 4N ammonium hydroxide at a pH of 5.5. The dissolved oxygen was controlled between 5 percent and 20 percent throughout the fermentation using agitation from 180 revolutions per minute to 480 revolutions per minute and airflow from 60 liters per minute to 100 liters per minute. Throughout the fermentation, 38.4 liters of an 850 grams per liter solution of 95 percent dextrose was fed to maintain a concentration less than 50 grams per liter. After 65 hours, the fermentor included 9,797 grams of biomass that included 6,056 grams of fatty acids. The resulting overall fatty acid productivity of the culture was 30.3 grams per liter per day and the resulting fatty acid yield (grams of fatty acid produced per grams of carbon feedstock) was 0.214.

### Example 6

In this example, the thraustochytrid Schizochytrium sp. (ATCC 20888) was cultivated in a 100 liter New Brunswick Scientific BioFlo 6000 fermentor with a carbon (glucose/fructose) and nitrogen (ammonium hydroxide) fed-batch process. The fermentation was inoculated with 6 liters of semicontinuous inoculum culture. For inoculum propagation a 14 liter VirTis fermentor was utilized. The inoculum medium included 10 liters of medium prepared in four separate groups. Group A included 98 grams MSG*1H₂O, 202 grams Na₂SO₄, 5 grams KCl, 22.5 grams MgSO₄*7H₂O, 23.1 grams (NH₄)₂SO₄, 14.7 grams KH₂PO₄, 0.9 grams CaCl₂*2H₂O, 17.7 milligrams MnCl₂*4H₂O, 18.1 milligrams ZnSO₄*7H₂O, 0.2 milligrams CoCl₂*6H₂O, 0.2 milligrams Na₂MoO₄*2H₂O, 11.8 milligrams CuSO₄*5H₂O, 11.8 milligrams NiSO₄*6H₂O, and 2 milliliters Dow 1520US (antifoam). Group A was autoclaved at 121 degrees Celsius in the inoculum fermentor at a volume of approximately 9.5 liters. Group B included 20 milliliters of a one liter stock solution containing 2.94 grams FeSO₄*7H₂O and 1 grams citric acid. The group B stock solution was autoclaved at 121 degrees Celsius. Group C included 37.6 milligrams thiamine-HCl, 1.9 milligrams vitamin B12, and 1.9 milligrams pantothenic acid hemi-calcium salt dissolved in 10 milliliters distilled water and filter sterilized. Group D included 1,000 milliliters of distilled water containing 400 grams glucose powder. After the fermentor was cooled to 29.5 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation.

The inoculum fermentor was inoculated with 18 milliliters of a standard ATCC 20888 shake flask culture and cultivated at 29.5 degrees Celsius, pH 5.5, 350 revolutions per minute agitation, and 8 liters per minute of air for a period of 23 hours, at which point 5 liters was harvested from the fermentor. To the remaining 5 liters of fermentation broth, 5 liters of freshly prepared media was added back to the inoculum fermentor. After an additional 5.3 hours of cultivation, 6 liters of inoculum broth were transferred to the 100 liter fermentor. The 100 liter fermentor included 80 liters of fermentation media. The fermentation media was prepared in a similar fashion to the inoculum fermentor. The fermentation media included 7 batched media groups. Group A included 1,089.6 grams Na₂SO₄, 57.6 grams K₂SO₄, 44.8 grams KCl, 181.6 grams MgSO₄*7H₂O, and 90.4 grams KH₂PO₄. Group A was steam sterilized at 122 degrees Celsius for 60 minutes in the 100 liter fermentor at a volume of approximately 35 liters. Group B included 90.4 grams (NH₄)₂SO₄ and 10.4 grams MSG*1H₂O in a volume of approximately 500 milliliters. Group C included 15.2 grams CaCl₂*2H₂O in a volume of approximately 200 milliliters. Group D included 600 grams of powdered glucose and 600 grams of powdered fructose in approximately 2 liters of distilled water. Group E included 248 milligrams MnCl₂*4H₂O, 248 milligrams ZnSO₄*7H₂O, 3.2 milligrams CoCl₂*6H₂O, 3.2 milligrams Na₂MoO₄*2H₂O, 165.6 milligrams CuSO₄*5H₂O, and 165.6 milligrams NiSO₄*6H₂O in a volume of approximately 1 liter. Group F included 824 milligrams FeSO₄*7H₂O and 280.3 milligrams citric acid in a volume of approximately 280 milliliters of distilled water. Group G included 780 milligrams thiamine-HCl, 12.8 milligrams vitamin B12, and 266.4 milligrams pantothenic acid hemi-calcium salt filter sterilized in a volume of approximately of 67.4 milliliters distilled water. Groups B, C, D, E, F, and G were combined and added to the fermentor after the fermentor reached an operating temperature of 29.5 degrees Celsius. The fermentor volume prior to inoculation was approximately 38 liters.

The fermentor was inoculated with 6 liters of broth from the fermentation described above. The fermentation was pH controlled utilizing a 5.4 liter solution of 4N ammonium hydroxide at a pH of 5.5. The dissolved oxygen was controlled between 2 percent and 35 percent throughout the fermentation using agitation from 180 revolutions per minute to 480 revolutions per minute and airflow from 60 liters per minute to 125 liters per minute. Throughout the fermentation, 37.9 liters of an 850 grams per liter solution of 50/50 fructose/dextrose was fed to maintain a concentration less than 100 grams per liter. After 95 hours, the fermentor included 8,575 grams of biomass that included 5,463 grams of fatty acids. This fed-batch process with glucose/fructose had a resulting fatty acid yield (grams of fatty acid produced per grams of carbon feedstock) of the culture of 0.14 and an overall fatty acid productivity of 17.3 grams per liter per day.

### Example 7

In this example, the thraustochytrid Schizochytrium sp. (ATCC 20888) was cultivated in a 10 liter New Brunswick Scientific BioFlo 3000 fermentor with a carbon (glucose/fructose) nonsterile fed-batch process. The fermentation was inoculated with 1.8 liters of culture. For inoculum propagation a 3 liter Broadley James (Irvine, Calif., U.S.A.) BioNet fermentor was utilized. The inoculum medium included 2 liters of medium prepared in six separate groups. Group A included 40.86 grams Na₂SO₄, 2.16 grams K₂SO₄, 1.68 grams KCl, 6.81 grams MgSO₄*7H₂O, and 3.39 grams KH₂PO₄. Group B included 0.57 grams CaCl₂*2H₂O. Group C included 11.3 grams (NH₄)₂SO₄ and 1.3 grams MSG*1H₂O. Group D included 103 milligrams FeSO₄*7H₂O, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O. Group D included 97.5 milligrams thiamine-HCl, 1.6 milligrams vitamin B12, and 33.3 milligrams pantothenic acid hemi-calcium salt dissolved in distilled water and filter sterilized. Group E included about 100 milliliters of distilled water containing 60 grams of a 50/50 glucose/fructose powder. After the fermentor was cooled to 29.5 degrees Celsius, groups B, C, D, and E were added to the fermentor. Using sodium hydroxide and sulfuric acid, the fermentor was pH adjusted to 5.5 and the dissolved oxygen was spanned to 100 percent prior to inoculation.

The inoculum fermentation was pH controlled utilizing a 0.678 liter solution of 4N ammonium hydroxide at a pH of 5.5. The inoculum fermentor was inoculated with 150 milliliters of a standard ATCC 20888 shake flask culture and cultivated at 29.5 degrees Celsius, pH 5.5 (w/NH₄OH), 636 revolutions per minute to 1,200 revolutions per minute agitation, and 0.8 liters per minute of air for a period of 55.25 hours, at which point 1.8 liters of inoculum broth were transferred to the 14 liter fermentor. The 14 liter fermentor included 10 liters of fermentation non sterile salt solution. The fermentation media was added to the fermentor under open air conditions without any sterilization of ingredients. The fermentation media included the following media components; 95.34 grams Na₂SO₄, 5.04 grams K₂SO₄, 3.92 grams KCl, 15.89 grams MgSO₄*7H₂O, 1.33 grams CaCl₂*2H₂O, and 90 grams of powdered 50/50 fructose/glucose in approximately 6 liters of distilled water.

After the fermentor reached a stable operating temperature of 29.5 degrees Celsius, the fermentor was inoculated with 1.8 liters of broth from the fermentation described above. The fermentation was not pH controlled. The dissolved oxygen was controlled to maintain a target of 20 percent throughout the fermentation using agitation from 300 revolutions per minute to 580 revolutions per minute and airflow from 6 liters per minute to 8 liters per minute. Throughout the fermentation, 1.845 liters of a heated nonsterile 850 grams per liter solution of 50/50 glucose/fructose was fed to maintain a total sugar concentration less than 120 grams per liter. After 62 hours, the fermentor included 847.8 grams of biomass that included 519.7 grams of fatty acids. The fatty acid yield (grams of fatty acid produced per grams of carbon feedstock) of this fermentation was 0.19 and the overall fatty acid productivity of 20.3 grams per liter per day. This two stage fermentation with a sterile growth stage and a nonsterile production stage with glucose/fructose had a substantial improvement in fatty acid production and fatty acid yield when compared to the standard fed-batch conditions (Example 6) that used a mixed feed of glucose/fructose.

### Example 8

In this example, the yeast Pseudozyma sp. (ATCC 11615), as confirmed by an independent taxonomic authority as greater than ninety-nine percent similar to a DNA match (DNA sequencing on the D1/D2 and ITS regions of the ribosomal genes) of both Pseudozyma aphidis and Pseudozyma rugulosa (where making a further distinction based on morphology may not be possible), was cultivated in a 14 liter New Brunswick Scientific BioFlo 3000 fermentor with a carbon (glucose) and nitrogen (ammonium hydroxide) fed-batch process. The fermentation was inoculated with 0.4 liters of inoculum culture. For inoculum propagation a 3 liter Broadley James BioNet fermentor was utilized. The inoculum medium included 2 liters of medium prepared in four separate groups. Group A included 18 grams MSG*1H₂O, 1.25 grams NaCl, 0.58 grams CaCl₂*2H₂O, 1 grams KCl, 10 grams MgSO₄*7H₂O, 0.74 grams (NH₄)₂SO₄, 6 grams yeast extract (T154), 1.04 grams KH₂PO₄, and 0.2 milliliters Dow 1520US (antifoam). Group A was autoclaved at 121 degrees Celsius in the inoculum fermentor at a volume of approximately 1.8 liters. Group B included 20.6 milligrams FeSO₄*7H₂O, 36.88 milligrams citric acid, 6.2 milligrams MnCl₂*4H₂O, 6.2 milligrams ZnSO₄*7H₂O, 0.08 milligrams CoCl₂*6H₂O, 0.08 milligrams Na₂MoO₄*2H₂O, 4.14 milligrams CuSO₄*5H₂O, and 4.14 milligrams NiSO₄*6H₂O in a volume of 9 milliliters of distilled water. The group B stock solution was autoclaved at 121 degrees Celsius. Group C included 19.5 milligrams thiamine-HCl, 0.32 milligrams vitamin B12, and 6.66 milligrams pantothenic acid hemi-calcium salt dissolved in 2 milliliters of distilled water and filter sterilized. Group D included 200 milliliters of distilled water containing 100 grams glucose powder. After the fermentor was cooled to 22.5 degrees Celsius, groups. B, C, and D were added to the fermentor. Using sodium hydroxide and sulfuric acid, the fermentor was pH adjusted to 6.9 and the dissolved oxygen was spanned to 100 percent prior to inoculation.

The inoculum fermentor was inoculated with 5 milliliters of a standard shake flask culture and cultivated at 22.5 degrees Celsius, pH 7, 430 revolutions per minute to 850 revolutions per minute agitation, and 0.5 liters to 1.0 liter per minute of air for a period of 26 hours, at which point 0.4 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 50 grams MSG*1H₂O, 6.25 grams NaCl, 20 grams Na₂SO₄, 2.9 grams CaCl₂*2H₂O, 10 grams KCl, 50 grams MgSO₄*7H₂O, 4.4 grams (NH₄)₂SO₄, 10 grams yeast extract (T154), 17.7 grams KH₂PO₄, and 1.0 milliliters Dow 1520US (antifoam). Group A was autoclaved at 121 degrees Celsius in the fermentor at a volume of approximately 5.5 liters. Group B included 103 milligrams FeSO₄*7H₂O, 184.4 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 36.5 micrograms biotin dissolved in 10 milliliters distilled water and filter sterilized. Group D included 500 milliliters of distilled water containing 300 grams glucose powder. After the fermentor was cooled to 22.5 degrees Celsius, groups B, C, and D were added to the fermentor. Using sodium hydroxide and sulfuric acid, the fermentor was pH adjusted to 6.5 and the dissolved oxygen was spanned to 100 percent prior to inoculation.

The fermentor volume prior to inoculation was approximately 5.5 liters. The fermentor was inoculated with 0.4 liters of broth from the fermentation described above. The fermentation was pH controlled utilizing a 0.883 liter solution of 4N ammonium hydroxide at a pH of 6.5. The dissolved oxygen was controlled to maintain a target set point of 20 percent throughout the fermentation using agitation from 290 revolutions per minute to 1,000 revolutions per minute and airflow at 9.5 liters per minute. Throughout the fermentation, 3.75 liters of an 850 grams per liter solution of dextrose was fed to maintain a concentration less than 60 grams per liter. After 95 hours, the fermentor included 1,172.7 grams of biomass that included 581.5 grams of fatty acids. The fatty acid yield (grams of fatty acid produced per grams of carbon feedstock) at 22.5 degrees Celsius, pH 6.5 and 1× nitrogen was 0.138, and the overall fatty acid productivity of 14.7 grams per liter per day.

### Example 9

In this example, the yeast Pseudozyma sp. (ATCC 11615) was cultivated in a 14 liter New Brunswick Scientific BioFlo 3000 fermentor with a carbon (sucrose) and nitrogen (ammonium hydroxide) fed-batch process. The fermentation was inoculated with 0.5 liters of inoculum culture. For inoculum propagation a 3 liter Broadley James BioNet fermentor was utilized. The inoculum medium included 2 liters of medium prepared in four separate groups. Group A included 18 grams MSG*1H₂O, 1.25 grams NaCl, 0.58 grams CaCl₂*2H₂O, 1 grams KCl, 10 grams MgSO₄*7H₂O, 0.74 grams (NH₄)₂SO₄, 6 grams yeast extract (T154), 1.04 grams KH₂PO₄, and 0.2 milliliters Dow 1520US (antifoam). Group A was autoclaved at 121 degrees in the fermentor at a volume of approximately 1.8 liters. Group B included 20.6 milligrams FeSO₄*7H₂O, 36.88 milligrams citric acid, 6.2 milligrams MnCl₂*4H₂O, 6.2 milligrams ZnSO₄*7H₂O, 0.08 milligrams CoCl₂*6H₂O, 0.08 milligrams Na₂MoO₄*2H₂O, 4.14 milligrams CuSO₄*5H₂O, and 4.14 milligrams NiSO₄*6H₂O in a volume of 9 milliliters. The group B stock solution was autoclaved at 121 degrees Celsius. Group C included 19.5 milligrams thiamine-HCl, 0.32 milligrams vitamin B12, and 6.66 milligrams pantothenic acid hemi-calcium salt dissolved in 2 milliliters distilled water and filter sterilized. Group D included 200 milliliters of distilled water containing 100 grams sucrose powder. After the fermentor was cooled to 29 degrees Celsius, groups B, C, and D were added to the fermentor. Using sodium hydroxide and sulfuric acid, the fermentor was pH adjusted to 6.5 and the dissolved oxygen was spanned to 100 percent prior to inoculation.

The inoculum fermentor was inoculated with 5 milliliters of a standard shake flask culture and cultivated at 29 degrees Celsius, pH 6.5, 634 revolutions per minute agitation, and 1.0 liters per minute of air for a period of 22.5 hours, at which point 0.5 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 50 grams MSG*1H₂O, 6.25 grams NaCl, 20 grams Na₂SO₄, 2.9 grams CaCl₂*2H₂O, 10 grams KCl, 50 grams MgSO₄*7H₂O, 4.4 grams (NH₄)₂SO₄, 10 grams yeast extract (T154), 17.7 grams KH₂PO₄, and 1.0 milliliters Dow 1520US (antifoam). Group A was autoclaved at 121 degrees Celsius in the fermentor at a volume of approximately 5.5 liters. Group B included 103 milligrams FeSO₄*7H₂O, 184.4 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of 45 milliliters. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 36.5 micrograms biotin dissolved in 10 milliliters and filter sterilized. Group D included 500 milliliters of distilled water containing 300 grams sucrose powder. After the fermentor was cooled to 29 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation. The fermentor volume prior to inoculation was approximately 4.8 liters.

The fermentor was inoculated with 0.5 liters of broth from the fermentation described above. The fermentation was pH controlled utilizing a 0.48 liter solution of 6N ammonium hydroxide at a pH of 6.5. The dissolved oxygen was controlled to maintain a target set point of 20 percent throughout the fermentation using agitation from 357 revolutions per minute to 833 revolutions per minute and airflow from 8 liters per minute to 13 liters per minute. Throughout the fermentation, 3.3 liters of an 850 grams per liter solution of sucrose was fed to maintain a total sugar (glucose+fructose+sucrose) concentration less than 90 grams per liter. After 70 hours, the fermentor included 984.9 grams of biomass that included 402.8 grams of fatty acids. The fatty acid yield (grams of fatty acid produced per grams of carbon feedstock) at 29.5 degrees Celsius, ph 5.5 and 1× nitrogen was 0.145, and the overall fatty acid productivity of 13.8 grams per liter per day.

### Example 10

In this example, the yeast Pseudozyma sp. (ATCC 11615) was cultivated in a 14 liter New Brunswick Scientific BioFlo 3000 fermentor with a carbon (sucrose) and nitrogen (ammonium hydroxide) fed-batch process. The fermentation was inoculated with 0.5 liters of inoculum culture. For inoculum propagation a 3 liter Broadley James BioNet fermentor was utilized. The inoculum medium included 2 liters of medium prepared in four separate groups. Group A included 18 grams MSG*1H₂O, 1.25 grams NaCl, 0.58 grams CaCl₂*2H₂O, 1 grams KCl, 10 grams MgSO₄*7H₂O, 0.74 grams (NH₄)₂SO₄, 6 grams yeast extract (T154), 1.04 grams KH₂PO₄, and 0.2 milliliters Dow 1520US (antifoam). Group A was autoclaved at 121 degrees in the inoculum fermentor at a volume of approximately 1.8 liters. Group B included 20.6 milligrams FeSO₄*7H₂O, 36.88 milligrams citric acid, 6.2 milligrams MnCl₂*4H2O, 6.2 milligrams ZnSO₄*7H2O, 0.08 milligrams CoCl₂*6H₂O, 0.08 milligrams Na₂MoO₄*2H₂O, 4.14 milligrams CuSO₄*5H₂O, and 4.14 milligrams NiSO₄*6H₂O in a volume of 9 milliliters of distilled water. The group B stock solution was autoclaved at 121 degrees Celsius. Group C included 19.5 milligrams thiamine-HCl, 0.32 milligrams vitamin B12, and 6.66 milligrams pantothenic acid hemi-calcium salt dissolved in 2 milliliters distilled water and filter sterilized. Group D included 200 milliliters of distilled water containing 100 grams sucrose powder. After the fermentor was cooled to 29 degrees Celsius, groups B, C, and D were added to the fermentor. Using sodium hydroxide and sulfuric acid, the fermentor was pH adjusted to 6.5 and the dissolved oxygen was spanned to 100 percent prior to inoculation.

The inoculum fermentor was inoculated with 5 milliliters of a standard shake flask culture and cultivated at 29 degrees Celsius, pH 6.5, 634 revolutions per minute agitation, and 1.0 liters per minute of air for a period of 22.5 hours, at which point 0.5 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 50 grams MSG*1H2O, 6.25 grams NaCl, 20 grams Na₂SO₄, 2.9 grams CaCl₂*2H₂O, 10 grams KCl, 50 grams MgSO₄*7H₂O, 4.4 grams (NH₄)₂SO₄, 10 grams yeast extract (T154), 17.7 grams KH2PO₄, and 1.0 milliliters Dow 1520US (antifoam). Group A was autoclaved at 121 degrees Celsius in the inoculum fermentor at a volume of approximately 5.5 liters. Group B included 103 milligrams FeSO₄*7H₂O, 184.4 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 36.5 micrograms biotin dissolved in 10 milliliters and filter sterilized. Group D included 500 milliliters of distilled water containing 300 grams sucrose powder. After the fermentor was cooled to 29 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation. The fermentor volume prior to inoculation was approximately 4.8 liters.

The fermentor was inoculated with 0.5 liters of broth from the fermentation described above. The fermentation was pH controlled utilizing a 0.28 liter solution of 6N ammonium hydroxide at a pH of 6.5. The dissolved oxygen was controlled to maintain a target set point of 20 percent throughout the fermentation using agitation from 357 revolutions per minute to 833 revolutions per minute and airflow from 8 liters per minute to 13 liters per minute. Throughout the fermentation, 3.7 liters of an 850 grams per liter solution of sucrose was fed to maintain a total sugar (glucose+fructose+sucrose) concentration less than 90 grams per liter. After 70 hours, the fermentor included 1,126.9 grams of biomass that included 535.8 grams of fatty acids. The final, cell density was 113 grams per liter dry weight. The fatty acid content was 47.5 percent of cellular dry weight, the average fatty acid productivity was 18.3 grams per liter per day and the resulting fatty acid yield (grams of fatty acids produced per grams of carbon feedstock) of the culture was 0.175. The highest peak fatty acid productivity of the culture, measured over a 6 hour period was 69.6 grams per liter per day. The highest peak fatty acid productivity of the culture measured over a 24 hour period was 50.4 grams per liter per day. The fatty acid yield (grams of fatty acid produced per grams of carbon feedstock consumed) at 29.5 degrees Celsius, pH 5.5 and 0.5× nitrogen was 0.175 gram per gram. The yield of fatty acid on oxygen (grams of fatty acid produced per grams of oxygen consumed) was 0.42 gram per gram. Examples 8, 9 and 10 demonstrate that fatty acid yield can be increased by using sucrose as the carbon source instead of glucose, increasing the fermentation temperature, decreasing the fermentation pH, and decreasing the nitrogen inputs. Table 1 below shows time versus measured variables during the fermentation.

TABLE 1

| | | | | Instantaneous | Total |
|---|---|---|---|---|---|
| | Fatty | | Fatty Acid | Fatty acid | Sucrose |
| | Acid | Biomass | Concentration | Production | Consumed |
| Time | (per- | (grams per | (grams per | (grams per | (grams per |
| (hours) | cent) | liter) | liter) | liter per day) | liter) |
| 0 | | | | | 31 |
| 11 | 8.76 | 13.87 | 0.73 | 1.57 | 31 |
| 19 | 14.00 | 55.06 | 4.93 | 13.10 | 54 |
| 22 | 19.07 | 71.81 | 8.21 | 22.83 | 77 |
| 27 | 29.03 | 85.91 | 16.21 | 42.17 | 137 |
| 30 | 35.60 | 93.53 | 23.64 | 54.04 | 173 |
| 35 | 40.48 | 108.32 | 37.27 | 69.06 | 173 |
| 43 | 46.60 | 117.42 | 49.25 | 35.93 | 228 |
| 46 | 47.00 | 122.65 | 49.58 | 2.30 | 241 |
| 54 | 48.55 | 130.66 | 52.65 | 9.22 | 278 |
| 63 | 51.35 | 135.19 | 55.54 | 8.11 | 308 |
| 70 | 49.71 | 134.15 | 56.01 | 1.56 | 341 |

### Example 11

In this example, the thraustochytrid Schizochytrium sp. (ATCC 20888) was cultivated in a 14 liter New Brunswick Scientific BioFlo 3000 fermentor with a primary (50/50 glucose/fructose) and secondary (glycerol) carbon feed and a nitrogen (ammonium hydroxide) fed-batch process. The fermentation was inoculated with 0.5 liters of semi-continuous inoculum culture. For inoculum propagation a 14 liter VirTis fermentor was utilized. The inoculum medium included 10 liters of medium prepared in four separate groups. Group A included 98 grams MSG*1H₂O, 202 grams Na₂SO₄, 5 grams KCl, 22.5 grams MgSO₄*7H₂O, 23.1 grams (NH₄)₂SO₄, 14.7 grams KH₂PO₄, 0.9 grams CaCl₂*2H₂O, 17.7 milligrams MnCl₂*4H₂O, 18.1 milligrams ZnSO₄*7H₂O, 0.2 milligrams CoCl₂*6H₂O, 0.2 milligrams Na₂MoO4*2H₂O, 11.8 milligrams CuSO₄*5H₂O, 11.8 milligrams NiSO₄*6H₂O, and 2 milliliters Dow 1520US (antifoam). Group A was autoclaved at 121 degrees Celsius in the inoculum fermentor at a volume of approximately 9.5 liters. Group B included 20 milliliters of a one liter stock solution containing 2.94 grams FeSO₄*7H₂O and 1 grams citric acid. The group B stock solution was autoclaved at 121 degrees Celsius. Group C included 37.6 milligrams thiamine-HCl, 1.9 milligrams vitamin B12, and 1.9 milligrams pantothenic acid hemi-calcium salt dissolved in 10 milliliters distilled water and filter sterilized. Group B included 1,000 milliliters of distilled water containing 400 grams glucose powder. After the fermentor was cooled to 29.5 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation.

The inoculum fermentor was inoculated with 18 milliliters of a standard ATCC 20888 shake flask culture and cultivated at 29.5 degrees Celsius, pH 5.6, 390 revolutions per minute agitation, and 8 liters per minute of air for a period of 24 hours, at which point 4 liters was harvested from the fermentor. To the remaining 6 liters of fermentation broth, 4 liters of freshly prepared media was added back to the inoculum fermentor.

After an additional 1.25 hours of cultivation, 0.5 liters of inoculum broth were transferred to the 14 liter fermentor. The 14 liter fermentor included 10 liters of fermentation media. The fermentation media was prepared in a similar fashion to the inoculum fermentor. The fermentation media included 7 batched media groups. Group A included 136.2 grams Na₂SO₄, 7.2 grams K₂SO₄, 5.6 grams KCl, 22.7 grams MgSO₄*7H₂O, and 11.3 grams KH₂PO₄. Group A was autoclaved at 121 degrees Celsius for about 80 minutes in the 14 liter fermentor at a volume of approximately 5 liters. Group B included 11.3 grams (NH₄)₂SO₄ and 1.3 grams MSG*1H₂O in a volume of approximately 200 milliliters. Group C included 1.9 grams CaCl₂*2H₂O in a volume of approximately 50 milliliters. Group D included 150 grams of powdered glucose/fructose in approximately 0.25 liters of distilled water. Group E included 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of approximately 0.2 liters. Group F included 103 milligrams FeSO₄*7H₂O and 35 milligrams citric acid in a volume of approximately 35 milliliters. Group G included 97.5 milligrams thiamine-HCl, 1.6 milligrams vitamin B12, and 33.3 milligrams pantothenic acid hemi-calcium salt filter sterilized in a volume of approximately 8.4 milliliters distilled water. Groups B, C, D, E, F, and G were combined and added to the fermentor after the fermentor reached an operating temperature of 29.5 degrees Celsius. The fermentor volume prior to inoculation was approximately 5 liters.

The fermentor was inoculated with 0.5 liters of broth from the semicontinuous fermentation described above. The fermentation was pH controlled utilizing a 0.678 liter solution of 4N ammonium hydroxide at a pH of 5.5. The dissolved oxygen was controlled above 0.1 percent throughout the fermentation using agitation from 250 revolutions per minute to 1,200 revolutions per minute and airflow from 7.5 liters per minute to 12 liters per minute. Throughout the fermentation, 2.8 liters of an 850 grams per liter solution of 50/50 glucose/fructose was fed to maintain a concentration less than 100 grams per liter. Simultaneously, 1.35 liters of a 625 grams per liter glycerol solution was fed to maintain a concentration between 2 and 20 grams per liter. After 91 hours, the fermentor included 1,433.6 grams of biomass that included 642.4 grams of fatty acid. In this fermentation, the fatty acid yield (grams of fatty acid produced per grams of carbon feedstock) was 0.186, and the overall fatty acid productivity was 24.3 grams per liter per day. When compared to the results of Example 6 (17.3 grams per liter per day overall fatty acid productivity), the present example demonstrates that under carbon and nitrogen fed-batch conditions, fatty acid productivity can be restored and enhanced by adding glycerol to the glucose/fructose feed matrix.

Fermentation Examples above, unless specified otherwise within this specification, can be generally followed along or conducted according to procedures from U.S. Pat. No. 6,607,900, unless specified within this specification.

### Example 12

The fatty acids from Examples 5 to 11 were analyzed for the fatty acid profiles as follows in Table 2. Data are expressed as weight percent of total fatty acids. Blank spaces within the table indicate a level below detection and/or characterization.

TABLE 2

| | Example percent | | | | | | |
|---|---|---|---|---|---|---|---|
| | 5 | 6 | 7 | 8 | 9 | 10 | 11 |
| 8:0 | | | | 0.3 | | | |
| 10:0 | | | | 0.8 | 0.3 | 3.4 | |
| 12:0 | 0.8 | | 0.5 | 0.6 | 0.3 | 2.7 | 0.5 |
| 13:0 | 0.2 | 0.1 | 0.1 | 0.1 | 0.1 | 0.2 | 0.1 |
| 14:0 | 23.0 | 19.8 | 18.7 | 0.8 | 1.0 | 1.1 | 18.8 |
| 15:1 | 0.5 | 0.5 | 0.3 | 0.8 | | | 0.2 |
| 16:0 | 38.8 | 40.1 | 33.5 | 17.6 | 22.9 | 17.8 | 41.8 |
| 16:1 | 2.4 | 1.1 | 12.3 | 3.8 | 0.4 | 2.2 | 1.3 |
| 17:0 | 0.1 | 0.8 | | 0.5 | | | |
| 18:0 | 0.9 | 1.3 | 0.8 | 4.9 | 27.2 | 4.6 | 1.1 |
| 18:1 | 0.8 | 0.4 | 4.3 | 47.1 | 34.6 | 47.5 | 0.7 |
| 18:2 | | | | 18.2 | 5.2 | 12.9 | |
| 18:3 n6 | 0.1 | 0.1 | | 0.7 | | | |
| 20:0 | 0.2 | 0.2 | | 1.6 | 3.3 | 2.1 | 0.2 |
| 20:1 | | | | 0.6 | | 0.4 | |
| 20:3 | 0.3 | 0.7 | | 0.2 | | | 0.2 |
| 20:4 | 0.9 | 0.7 | | 0.7 | | | |
| 20:5 | 0.4 | 0.4 | 0.4 | | | | 0.4 |
| 22:0 | 0.1 | 0.1 | | 1.5 | 2.4 | 1.6 | |
| 22:4 | 0.2 | 0.8 | | | | | |
| 22:5 | 8.0 | 9.7 | 8.4 | | | | 9.6 |
| 22:6 | 21.2 | 23.2 | 20.8 | | | | 24.4 |
| 24:0 | | | | 1.0 | 1.1 | 0.7 | |
| fat | 63.1 | 63.7 | 61.4 | 49.6 | 40.9 | 47.5 | 63.9 |
| unknown | 1.3 | 2.3 | 0.3 | 0.7 | 0.2 | 2.3 | 0.6 |

### Example 13

In this example, the yeast Rhodosporidium toruloides (CBS6016) was cultivated in a 14 liter New Brunswick Scientific BioFlo 3000 fermentor with a carbon (sucrose) and nitrogen (ammonium hydroxide) fed-batch process. The fermentation was inoculated with 0.5 liters of inoculum culture. For inoculum propagation a 3 liter Broadley James BioNet fermentor was utilized. The inoculum medium included 2.3 liters of medium prepared in four separate groups. Group A included 20.7 grams MSG*1H₂O, 1.44 grams NaCl, 0.667 grams CaCl₂*2H₂O, 2.3 grams KCl, 11.5 grams MgSO₄*7H₂O, 0.85 grams (NH₄)₂SO₄, 13.8 grams yeast extract (T154), 1.196 grams KH₂PO₄, and 0.23 milliliters Dow 1520US (antifoam). Group A was autoclaved at 121 degrees in the inoculum fermentor at a volume of approximately 2.1 liters. Group B included 23.7 milligrams FeSO₄*7H₂O, 42.412 milligrams citric acid, 7.13 milligrams MnCl₂*4H2O, 7.13 milligrams ZnSO₄*7H2O, 0.092 milligrams CoCl₂*6H₂O, 0.092 milligrams Na₂MoO₄*2H₂O, 4.761 milligrams CuSO₄*5H₂O, and 4.761 milligrams NiSO₄*6H₂O in a volume of 4.5 milliliters of distilled water. The group B stock solution was autoclaved at 121 degrees Celsius. Group C included 22.425 milligrams thiamine-HCl, 0.368 milligrams vitamin B12, and 7.67 milligrams pantothenic acid hemi-calcium salt dissolved in approximately 2 milliliters distilled water and filter sterilized. Group D included 200 milliliters of distilled water containing 115 grams sucrose powder. After the fermentor was cooled to 30 degrees Celsius, groups B, C, and D were added to the fermentor. Using sodium hydroxide and sulfuric acid, the fermentor was pH adjusted to 6.9 and the dissolved oxygen was spanned to 100 percent prior to inoculation.

The inoculum fermentor was inoculated with 10 milliliters of a standard shake flask culture and cultivated at 30 degrees Celsius, pH 6.9, 636 revolutions per minute agitation, and 1.2 liters per minute of air for a period of 16.75 hours, at which point 0.5 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 6.25 grams NaCl, 20 grams Na₂SO₄, 2.9 grams CaCl₂*2H₂O, 10 grams KCl, 50 grams MgSO₄*7H₂O, 4.2 grams (NH₄)₂SO₄, 10 grams yeast extract (T154), 17.65 grams KH2PO4, and 1.0 milliliters Dow 1520US (antifoam). Group A was autoclaved at 121 degrees Celsius in the fermentor at a volume of approximately 6.25 liters. Group B included 103 milligrams FeSO₄*7H₂O, 370 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of approximately 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 500 milliliters of distilled water containing 300 grams sucrose powder. After the fermentor was cooled to 27 degrees Celsius, groups B, C, and D were added to the fermentor. Using sodium hydroxide and sulfuric acid, the fermentor was pH adjusted to 5.5 and the dissolved oxygen was spanned to 100 percent prior to inoculation. The fermentor volume prior to inoculation was approximately 5.6 liters.

The fermentor was inoculated with 0.5 liters of broth from the inoculum fermentation described above. The fermentation was pH controlled utilizing a 0.26 liter solution of 6N ammonium hydroxide at a pH of 5.5. The dissolved oxygen was controlled to maintain a target set point of 20 percent throughout the fermentation using agitation from 357 revolutions per minute to 1200 revolutions per minute, airflow from 4 liters per minute to 8 liters per minute, and oxygen from 0.0 liters per minute to 4.0 liters per minute. Throughout the fermentation, 3.85 liters of an 850 grams per liter solution of sucrose was fed to maintain a total sugar (glucose+fructose+sucrose) concentration less than 65 grams per liter. After 89 hours, the fermentor included 1127 grams of biomass that included 610.8 grams of fatty acids. The final cell density was 112.7 grams per liter dry weight. The fatty acid content was 54.18 percent of cellular dry weight, the average fatty acid productivity was 16.56 grams per liter per day and the resulting fatty acid yield (grams of fatty acids produced per grams of carbon feedstock) of the culture was 0.1956. The highest peak fatty acid productivity of the culture, measured over an 8 hour period was 31.68 grams per liter per day. The highest peak fatty acid productivity of the culture measured over a 24 hour period was 30.1 grams per liter per day. The fatty acid yield (grams of fatty acid produced per grams of carbon feedstock) at 27 degrees Celsius, pH 5.5 and 0.5× nitrogen was 0.1956.

Lipid extraction was conducted by mixing 500 grams to 600 grams of freeze dried biomass with 6× mass to volume of hexane. The solids were wetted in using a high shear mixer. The mixture was passed twice through a Microfluidics (Newton, Mass., U.S.A.) homogenizer (110Y) set-up for cell disruption by using of a Z type interaction chamber (G10Z) at pressures of 10,000 psi. The homogenized material was centrifuged at 4,000 revolutions per minute for 5 minutes to separate the solids from the hexane/crude oil layer. The solids, representing the hexane extracted biomass is referred to as biomeal. The lighter hexane crude oil layer was removed and the hexane evaporated in a Rotary Evaporator (Buchi Labortechnik AG, Flawil, Switzerland) with a water bath temperature of about 50 degrees Celsius to about 60 degrees Celsius. The oil was purged with nitrogen and stored refrigerated. Samples of starting freeze dried biomass and biomeal following hexane extraction were submitted for FAME analysis. Extraction yields are based on total fatty acids in the starting biomass minus the total fatty acids in the biomeal divided by the total fatty acids in the starting biomass. The extraction yield was 91 percent.

Overall indices for fatty acid efficiency are as follows: OILE=2170, OILE1=3.0, OILE2=180, OILE3=5.4 and OILE4=72.1, based on an assumed value for G of 0.4 (yield of fatty acids on oxygen expressed as grams of fatty acids produced per gram of oxygen).

### Example 14

In this example, the yeast Rhodosporidium toruloides (CBS8587) was cultivated in a 14 liter New Brunswick Scientific BioFlo 3000 fermentor with a carbon (sucrose) and nitrogen (ammonium hydroxide) fed-batch process. The fermentation was inoculated with 0.5 liters of inoculum culture. For inoculum propagation a 3 liter Broadley James BioNet fermentor was utilized. The inoculum medium included 2.3 liters of medium prepared in four separate groups. Group A included 20.7 grams MSG*1H₂O, 1.44 grams NaCl, 0.667 grams CaCl₂*2H₂O, 2.3 grams KCl, 11.5 grams MgSO₄*7H₂O, 0.85 grams (NH₄)₂SO₄, 13.8 grams yeast extract (T154), 1.196 grams KH₂PO₄, and 0.23 milliliters Dow 1520US (antifoam). Group A was autoclaved at 121 degrees in the inoculum fermentor at a volume of approximately 2.15 liters. Group B included 23.7 milligrams FeSO₄*7H₂O, 42.412 milligrams citric acid, 7.13 milligrams MnCl₂*4H2O, 7.13 milligrams ZnSO₄*7H2O, 0.092 milligrams CoCl₂*6H₂O, 0.092 milligrams Na₂MoO₄*2H₂O, 4.761 milligrams CuSO₄*5H₂O, and 4.761 milligrams NiSO₄*6H₂O in a volume of 52 milliliters of distilled water. The group B stock solution was autoclaved at 121 degrees Celsius. Group C included 22.425 milligrams thiamine-HCl, 0.368 milligrams vitamin B12, and 7.67 milligrams pantothenic acid hemi-calcium salt dissolved in approximately 2 milliliters distilled water and filter sterilized. Group D included 200 milliliters of distilled water containing 115 grams sucrose powder. After the fermentor was cooled to 27 degrees Celsius, groups B, C, and D were added to the fermentor. Using sodium hydroxide and sulfuric acid, the fermentor was pH adjusted to 6.91 and the dissolved oxygen was spanned to 100 percent prior to inoculation.

The inoculum fermentor was inoculated with 26 milliliters of a standard shake flask culture and cultivated at 27 degrees Celsius, pH 6.9, 643 revolutions per minute agitation, and 1.2 liters per minute of air for a period of 16.75 hours, at which point 0.5 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 6.25 grams NaCl, 20 grams Na₂SO₄, 2.9 grams CaCl₂*2H₂O, 10 grams KCl, 50 grams MgSO₄*7H₂O, 4.2 grams (NH₄)₂SO₄, 10 grams yeast extract (T154), 17.65 grams KH2PO4, and 1.0 milliliters Dow 1520US (antifoam). Group A was autoclaved at 121 degrees Celsius in the fermentor at a volume of approximately 6.5 liters. Group B included 103 milligrams FeSO₄*7H₂O, 370 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of approximately 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 500 milliliters of distilled water containing 300 grams sucrose powder. After the fermentor was cooled to 27 degrees Celsius, groups B, C, and D were added to the fermentor. Using sodium hydroxide and sulfuric acid, the fermentor was pH adjusted to 7 and the dissolved oxygen was spanned to 100 percent prior to inoculation. The fermentor volume prior to inoculation was approximately 6.3 liters.

The fermentor was inoculated with 0.5 liters of broth from the inoculum fermentation described above. The fermentation was pH controlled utilizing a 0.26 liter solution of 6N ammonium hydroxide at a pH of 6.9. The dissolved oxygen was controlled to maintain a target set point of 20 percent throughout the fermentation using agitation from 357 revolutions per minute to 1200 revolutions per minute, airflow from 4 liters per minute to 9 liters per minute, and oxygen from 0.0 liters per minute to 4.0 liters per minute. Throughout the fermentation, 4.13 liters of an 850 grams per liter solution of sucrose was fed to maintain a total sugar (glucose+fructose+sucrose) concentration less than 75 grams per liter. After 92 hours, the fermentor included 1334 grams of biomass that included 701 grams of fatty acids. The final cell density was 133.4 grams per liter dry weight. The fatty acid content was 54 percent of cellular dry weight, the average fatty acid productivity was 18.75 grams per liter per day and the resulting fatty acid yield (grams of fatty acids produced per grams of carbon feedstock) of the culture was 0.1988. The highest peak fatty acid productivity of the culture, measured over an 8 hour period was 39 grams per liter per day. The highest peak fatty acid productivity of the culture measured over a 24 hour period was 26 grams per liter per day. The fatty acid yield (grams of fatty acid produced per grams of carbon feedstock) at 27 degrees Celsius, pH 6.9 and 0.5× nitrogen was 0.1988.

Lipid extraction was conducted as in Example 13. The extraction yield was 78 percent.

Overall indices for fatty acid efficiency are as follows: OILE=2040, OILE1=2.7, OILE2=196, OILE3=3.8 and OILE4=78.5, based on an assumed value for G of 0.4 (yield of fatty acids on oxygen expressed as grams of fatty acids produced per gram of oxygen).

### Example 15

In this example, a strain of the yeast Sporidiobolus pararoseus (ATCC 11616), as confirmed as a one hundred percent DNA match to several strains of Sporidiobolus pararoseus, was cultivated in a 14 liter New Brunswick Scientific BioFlo 3000 fermentor with a carbon (sucrose syrup) and nitrogen (ammonium hydroxide) fed-batch process. The fermentation was inoculated with 0.75 liters of inoculum culture. For inoculum propagation a 3 liter Broadley James BioNet fermentor was utilized. The inoculum medium included 2.3 liters of medium prepared in four separate groups. Group A included 20.7 grams MSG*1H₂O, 1.44 grams NaCl, 0.667 grams CaCl₂*2H₂O, 2.3 grams KCl, 11.5 grams MgSO₄*7H₂O, 0.85 grams (NH₄)₂SO₄, 13.8 grams yeast extract (T154), 1.196 grams KH₂PO₄, and 0.23 milliliters Dow 1520US (antifoam). Group A was autoclaved at 121 degrees in the inoculum fermentor at a volume of approximately 2.1 liters. Group B included 23.7 milligrams FeSO₄*7H₂O, 42.412 milligrams citric acid, 7.13 milligrams MnCl₂*4H2O, 7.13 milligrams ZnSO₄*7H2O, 0.092 milligrams CoCl₂*6H₂O, 0.092 milligrams Na₂MoO₄*2H₂O, 4.761 milligrams CuSO₄*5H₂O, and 4.761 milligrams NiSO₄*6H₂O in a volume of 4.5 milliliters of distilled water. The group B stock solution was autoclaved at 121 degrees Celsius. Group C included 22.425 milligrams thiamine-HCl, 0.368 milligrams vitamin B12, and 7.67 milligrams pantothenic acid hemi-calcium salt dissolved in approximately 2 milliliters distilled water and filter sterilized. Group D included 200 milliliters of distilled water containing 115 grams sucrose powder. After the fermentor was cooled to 30 degrees Celsius, groups B, C, and D were added to the fermentor. Using sodium hydroxide and sulfuric acid, the fermentor was pH adjusted to 6.9 and the dissolved oxygen was spanned to 100 percent prior to inoculation.

The inoculum fermentor was inoculated with 29.5 milliliters of a standard shake flask culture and cultivated at 30 degrees Celsius, pH 6.9, 847 revolutions per minute agitation, and 1.2 liters per minute of air for a period of 14.5 hours, at which point 0.75 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 6.25 grams NaCl, 4.2 grams (NH₄)₂SO₄, 10 grams yeast extract (T154), 12.66 grams Na₂HPO₄, and 1.0 milliliters Dow 1520US (antifoam). Group A was autoclaved at 121 degrees Celsius in the fermentor at a volume of approximately 6.25 liters. Group B included 103 milligrams FeSO₄*7H₂O, 370 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of approximately 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 700 milliliters of sugar syrup obtained from Raceland Raw Sugar Corporation in Louisiana, U.S.A. After the fermentor was cooled to 27 degrees Celsius, groups B, C, and D were added to the fermentor. Using sodium hydroxide and sulfuric acid, the fermentor was pH adjusted to 7 and the dissolved oxygen was spanned to 100 percent prior to inoculation. The fermentor volume prior to inoculation was approximately 6.15 liters.

The fermentor was inoculated with 0.75 liters of broth from the inoculum fermentation described above. The fermentation was pH controlled utilizing a 0.26 liter solution of 6N ammonium hydroxide at a pH of 7. The dissolved oxygen was controlled to maintain a target set point of 20 percent throughout the fermentation using agitation from 357 revolutions per minute to 1200 revolutions per minute, airflow from 3 liters per minute to 8 liters per minute, and oxygen from 0 liters per minute to 5 liters per minute. Throughout the fermentation, 5.65 liters of (Raceland) sugar syrup was fed to maintain a total sugar (glucose+fructose+sucrose) concentration less than 80 grams per liter. After 92 hours, the fermentor included 1251 grams of biomass that included 670.7 grams of fatty acids. The final cell density was 125.1 grams per liter dry weight. The fatty acid content was 53.63 percent of cellular dry weight, the average fatty acid productivity was 17.56 grams per liter per day and the resulting fatty acid yield (grams of fatty acids produced per grams of carbon feedstock) of the culture was 0.1625. The highest peak fatty acid productivity of the culture, measured over an 8 hour period was 56.8 grams per liter per day. The highest peak fatty acid productivity of the culture measured over a 24 hour period was 30.5 grams per liter per day. The fatty acid yield (grams of fatty acid produced per grams of carbon feedstock) at 27 degrees Celsius, pH 7 and 0.5× nitrogen was 0.1625.

### Example 16

In this example, the yeast Rhodotorula ingenosa (ATCC 11617), as confirmed as a ninety-nine percent DNA match to Rhodotorula ingenosa, was cultivated in a 14 liter New Brunswick Scientific BioFlo 3000 fermentor with a carbon (sucrose syrup) and nitrogen (ammonium hydroxide) fed-batch process. The fermentation was inoculated with 0.75 liters of inoculum culture. For inoculum propagation a 3 liter Broadley James BioNet fermentor was utilized. The inoculum medium included 2.2 liters of medium prepared in four separate groups. Group A included 20.7 grams MSG*1H₂O, 1.44 grams NaCl, 0.667 grams CaCl₂*2H₂O, 2.3 grams KCl, 11.5 grams MgSO₄*7H₂O, 0.85 grams (NH₄)₂SO₄, 13.8 grams yeast extract (T154), 1.196 grams KH₂PO₄, and 0.23 milliliters Dow 1520US (antifoam). Group A was autoclaved at 121 degrees in the inoculum fermentor at a volume of approximately 2.15 liters. Group B included 23.7 milligrams FeSO₄*7H₂O, 42.412 milligrams citric acid, 7.13 milligrams MnCl₂*4H2O, 7.13 milligrams ZnSO₄*7H2O, 0.092 milligrams CoCl₂*6H₂O, 0.092 milligrams Na₂MoO₄*2H₂O, 4.761 milligrams CuSO₄*5H₂O, and 4.761 milligrams NiSO₄*6H₂O in a volume of 52 milliliters of distilled water. The group B stock solution was autoclaved at 121 degrees Celsius. Group C included 22.425 milligrams thiamine-HCl, 0.368 milligrams vitamin B12, and 7.67 milligrams pantothenic acid hemi-calcium salt dissolved in approximately 2 milliliters distilled water and filter sterilized. Group D included 200 milliliters of distilled water containing 115 grams sucrose powder. After the fermentor was cooled to 27 degrees Celsius, groups B, C, and D were added to the fermentor. Using sodium hydroxide and sulfuric acid, the fermentor was pH adjusted to 5.09 and the dissolved oxygen was spanned to 100 percent prior to inoculation.

The inoculum fermentor was inoculated with 23 milliliters of a standard shake flask culture and cultivated at 27 degrees Celsius, pH 5.09, 644 revolutions per minute agitation, and 1.2 liters per minute of air for a period of 17.17 hours, at which point 0.75 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 6.25 grams NaCl, 4.2 grams (NH₄)₂SO₄, 10 grams yeast extract (T154), 12.66 grams Na₂HPO₄, and 1.0 milliliters Dow 1520US (antifoam). Group A was autoclaved at 121 degrees Celsius in the fermentor at a volume of approximately 6.25 liters. Group B included 103 milligrams FeSO₄*7H₂O, 370 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of approximately 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 700 milliliters of sugar syrup obtained from Raceland Sugar in Louisiana. After the fermentor was cooled to 27 degrees Celsius, groups B, C, and D were added to the fermentor. Using sodium hydroxide and sulfuric acid, the fermentor was pH adjusted to 5 and the dissolved oxygen was spanned to 100 percent prior to inoculation. The fermentor volume prior to inoculation was approximately 4.85 liters.

The fermentor was inoculated with 0.75 liters of broth from the inoculum fermentation described above. The fermentation was pH controlled utilizing a 0.27 liter solution of 6N ammonium hydroxide at a pH of 5. The dissolved oxygen was controlled to maintain a target set point of 20 percent throughout the fermentation using agitation from 357 revolutions per minute to 1100 revolutions per minute, airflow from 0.9 liters per minute to 7.9 liters per minute, and oxygen from 0 liters per minute to 7 liters per minute. Throughout the fermentation, 4.9 liters of (Raceland) sugar syrup was fed to maintain a total sugar (glucose+fructose+sucrose) concentration less than 75 grams per liter. After 89 hours, the fermentor included 1104 grams of biomass that included 553 grams of fatty acids. The final cell density was 110.4 grams per liter dry weight. The fatty acid content was 50.1 percent of cellular dry weight, the average fatty acid productivity was 14.96 grams per liter per day and the resulting fatty acid yield (grams of fatty acids produced per grams of carbon feedstock) of the culture was 0.1634. The highest peak fatty acid productivity of the culture, measured over an 8 hour period was 31.9 grams per liter per day. The highest peak fatty acid productivity of the culture measured over a 24 hour period was 29.9 grams per liter per day. The fatty acid yield (grams of fatty acid produced per grams of carbon feedstock) at 27 degrees Celsius, pH 5 and 0.5× nitrogen was 0.1634.

### Example 17

In this example, the green algae Chlorella protothecoides UTEX 250 (MK28415) was cultivated in a 14 liter New Brunswick Scientific BioFlo 3000 fermentor with a carbon (acid hydrolyzed sucrose syrup) and nitrogen (ammonium hydroxide) fed-batch process. The fermentation was inoculated with 1 liter of inoculum culture. For inoculum propagation a 14 liter Broadley James BioNet fermentor was utilized. The inoculum medium included 10 liters of medium prepared in five separate groups. Group A included 20 grams MSG*1H₂O, 2.9 grams CaCl₂*2H₂O, 10 grams yeast extract (T154), and 1.0 milliliters Dow 1520US (antifoam). Group A was autoclaved at 121 degrees in the inoculum fermentor at a volume of approximately 9.5 liters. Group B included 20 grams KH₂PO₄ in a volume of approximately 200 milliliters. The group B stock solution was autoclaved at 121 degrees Celsius. Group C included 103 milligrams FeSO₄*7H₂O, 184.4 milligrams citric acid, 18.1 milligrams MnCl₂*4H2O, 2.2 milligrams ZnSO₄*7H2O, 0.49 milligrams CoCl₂*6H₂O, 3.9 milligrams Na₂MoO₄*2H₂O, 28.6 milligrams H₃BO₃, and 0.79 milligrams CuSO₄*5H₂O all dissolved in distilled water. The group C stock solution was autoclaved at 121 degrees Celsius. Group D included 97.5 milligrams thiamine-HCl, 1.6 milligrams vitamin B12, and 33.3 milligrams pantothenic acid hemi-calcium salt dissolved in approximately 20 milliliters distilled water and filter sterilized. Group E included 1000 milliliters of distilled water containing 500 grams corn syrup. After the fermentor was cooled to 27 degrees Celsius, groups B, C, D, and E were added to the fermentor. Using sodium hydroxide and sulfuric acid, the fermentor was pH adjusted to 7 and the dissolved oxygen was spanned to 100 percent prior to inoculation.

The inoculum fermentor was inoculated with 400 milliliters of a standard shake flask culture and cultivated at 27 degrees Celsius, pH 7, 384 revolutions per minute agitation, and 5 liters per minute of air for a period of 43.5 hours, at which point 1 liter was harvested from the fermentor and transferred to the 14 liter fermentor. The 14 liter fermentor included 10 liters of fermentation media. The fermentation media was prepared in a similar fashion to the inoculum fermentor. The fermentation media included 5 batched media groups. Group A included 25 grams MSG*1H₂O, 2.9 grams CaCl₂*2H₂O, 10 grams yeast extract (T154), and 1.0 milliliters Dow 1520US (antifoam). Group A was autoclaved at 121 degrees Celsius in the fermentor at a volume of approximately 5.5 liters. Group B included 25 grams KH₂PO₄ in a volume of approximately 250 milliliters. The group B stock solution was autoclaved at 121 degrees Celsius. Group C included 257.5 milligrams FeSO₄*7H₂O, 461 milligrams citric acid, 45.25 milligrams MnCl₂*4H2O, 5.55 milligrams ZnSO₄*7H₂O, 1.225 milligrams CoCl₂*6H₂O, 9.75 milligrams Na₂MoO₄*2H₂O, 71.5 milligrams H₃BO₃, and 1.975 milligrams CuSO₄*5H₂O all dissolved in distilled water. The group C stock solution was autoclaved at 121 degrees Celsius. Group D included 35.8 micrograms biotin, 97.5 milligrams thiamine-HCl, 1.6 milligrams vitamin B12, and 33.3 milligrams pantothenic acid hemi-calcium salt dissolved in approximately 20 milliliters distilled water and filter sterilized. Group E included approximately 1000 milliliters of hydrolyzed sugar syrup obtained from Raceland Sugar in Louisiana. The sugar syrup was hydrolyzed by adding sulfuric acid to a pH of about 4 and sterilizing at 121 degrees Celsius for at least one hour. After the fermentor was cooled to 27 degrees Celsius, groups B, C, D, and E were added to the fermentor. Using sodium hydroxide and sulfuric acid, the fermentor was pH adjusted to 7 and the dissolved oxygen was spanned to 100 percent prior to inoculation. The fermentor volume prior to inoculation was approximately 5.9 liters.

The fermentor was inoculated with 1 liter of broth from the inoculum fermentation described above. The fermentation was pH controlled utilizing a 0.27 liter solution of 6N ammonium hydroxide at a pH of 7 until the ammonium hydroxide feed was exhausted (approximately 55 hours after inoculation), at which point 4N sodium hydroxide was used for the remainder of the fermentation. The dissolved oxygen was controlled to maintain a target set point of 20 percent throughout the fermentation using agitation from 357 revolutions per minute to 850 revolutions per minute and airflow at 8 liters per minute. Throughout the fermentation, 5.2 liters of (Raceland) sugar syrup was fed to maintain a total sugar (glucose+fructose+sucrose) concentration less than 55 grams per liter. After 93 hours, the fermentor included 1082 grams of biomass that included 462.4 grams of fatty acids. The final cell density was 108.2 grams per liter dry weight. The fatty acid content was 42.73 percent of cellular dry weight, the average fatty acid productivity was 11.93 grams per liter per day and the resulting fatty acid yield (grams of fatty acids produced per grams of carbon feedstock) of the culture was 0.17617. The highest peak fatty acid productivity of the culture, measured over an 8 hour period was 33.6 grams per liter per day. The highest peak fatty acid productivity of the culture measured over a 24 hour period was 24.4 grams per liter per day. The fatty acid yield (grams of fatty acid produced per grams of carbon feedstock) at 27 degrees Celsius, pH 7 and 0.5× nitrogen was 0.17617.

As used herein the terms “has”, “having”, “comprising” “with”, “containing”, and “including” are open and inclusive expressions. Alternately, the term “consisting” is a closed and exclusive expression. Should any ambiguity exist in construing any term in the claims or the specification, the intent of the drafter is toward open and inclusive expressions.

As used herein the term “and/or the like” provides support for any and all individual and combinations of items and/or members in a list, as well as support for equivalents of individual and combinations of items and/or members.

Regarding an order, number, sequence, omission, and/or limit of repetition for steps in a method or process, the drafter intends no implied order, number, sequence, omission, and/or limit of repetition for the steps to the scope of the invention, unless explicitly provided.

Regarding ranges, ranges are to be construed as including all points between upper values and lower values, such as to provide support for all possible ranges contained between the upper values and the lower values including ranges with no upper bound and/or lower bound.

Basis for operations, percentages, and procedures can be on any suitable basis, such as a mass basis, a volume basis, a mole basis, and/or the like. If a basis is not specified, a mass basis or other appropriate basis should be used.

It will be apparent to those skilled in the art that various modifications and variations can be made in the disclosed structures and methods without departing from the scope or spirit of the invention. Particularly, descriptions of any of the embodiments can be freely combined with descriptions of other embodiments to result in combinations and/or variations of two or more elements and/or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. An isolated organism for producing a biological oil, the organism comprising an overall index for fatty acid efficiency one (OILE1) of at least about 5.1, wherein:
OILE1=C*D*F; and
C=a fatty acid productivity in grams per liter per day;
D=a fatty acid yield in grams of fatty acids produced per grams of feedstock consumed; and
F=an extraction efficiency on a percent of total fatty acid content basis.

2. The isolated organism of claim 1, wherein the isolated organism has a cell density of at least about 115 grams per liter.

3. The isolated organism of any of claims 1-2, wherein the isolated organism has a fatty acid content of at least about 49 percent on a dry mass basis.

4. The isolated organism of any of claims 1-3, wherein the isolated organism has a 24 hour peak fatty acid productivity of at least about 30 grams per liter per clay.

5. The isolated organism of any of claims 1-4, wherein the isolated organism has a yield of fatty acid on oxygen of more than about 0.4 grams of fatty acids produced per gram of oxygen consumed.

6. The isolated organism of any of claims 1-5, wherein the isolated organism comprises Pseudozyma aphidis, Pseudozyma rugulosa, Pseudozyma sp., Rhodosporidium fluviale, Rhodosporidium paludigenum, Rhodotorula glutinis, Rhodotorula hordea, Rhodotorula ingenosa, Sporobolomyces ruberrimus, Tremella sp., Ustilago sp., Rhodosporidium toruloides, Sporidiobolus pararoseus, Leucosporidium scottii, Pseudozyma antarctica, Rhodosporidium sphaerocarpum, Rhodotorula muscorum, Cryptococcus laurentii, Candida tropicalis, Rhodosporidium diobovatum, Chlorella protothecoides, or combinations thereof.

7. The isolated organism of any claims of 1-5, wherein the organism has the identifying characteristics of ATCC Patent Deposit Designation of PTA-11615 and mutant strains derived therefrom.

8. The isolated organism of any claims of 1-5, wherein the organism has the identifying characteristics of ATCC Patent Deposit Designation of PTA-11616 and mutant strains derived therefrom.

9. The isolated organism of any claim of 1-5, wherein the organism has the identifying characteristics of ATCC Patent Deposit Designation of PTA-11617 and mutant strains derived therefrom.

10. A biological oil or a biofuel comprising or made from fatty acids made from the isolated organism of any of claims 1-9.

11. A method of producing a biological oil, the method comprising:
producing an organism comprising fatty acids; and
removing the fatty acids from the organism to form the biological oil;
wherein the organism meets or exceeds at least two metrics, each metric comprising:
a cell density of at least about 115 grams per liter;
a fatty acid content of at least about 49 percent on a dry mass basis;
a fatty acid productivity of at east about 15 grams per liter per day;
a fatty acid yield of at least about 0.175 grams of fatty acids produced per grams of feedstock consumed;
a 24 hour peak fatty acid productivity of at least about 30 grams per liter per day; or
a yield of fatty acid on oxygen of more than about 0.4 grams of fatty acids produced per gram of oxygen consumed.

12. The method of claim 11, wherein the organism meets or exceeds at least three more of the metrics.

13. The method of any of claims 11-12, wherein the organism comprises organisms of a genus of Rhodosporidium, Pseudozyma, Tremella, Rhodotorula Sporidiobolus, Sporobolomyces, Ustilago, Cryptococcus, Leucosporidium, Candida, or combinations thereof.

14. The method of any of claims 11-2, wherein the organism comprises organisms of a genus of Schizochytrium, Thraustochytrium, Ulkenia, Chlorella, Prototheca, or combinations thereof.

15. The method of any of claims 11-14, further comprising a feedstock wherein the feedstock comprises at least one organic acid.

16. A biological oil or biofuel comprising or made from fatty acids made by the method of any of claims 11-14.
